(19) United States
(12) Patent Application Publication
(10) Pub. No.: US 2022/0099677 A1
(43) Pub. Date: Mar. 31, 2022

(54) METHODS FOR DETECTING AND QUANTIFYING MEMBRANE-ASSOCIATED PROTEINS

(71) Applicant: GENENTECH, INC., South San Francisco, CA (US)

(72) Inventors: Vinita GUPTA, South San Francisco, CA (US); Ann Therese BRADY, South San Francisco, CA (US); James Thomas KOERBER, South San Francisco, CA (US); Xiangdan WANG, South San Francisco, CA (US); Minh Michael Nhu PHAN, South San Francisco, CA (US); Yonglian SUN, South San Francisco, CA (US)

(73) Assignee: GENENTECH, INC., South San Francisco, CA (US)

(21) Appl. No.: 17/469,533

(22) Filed: Sep. 8, 2021

Related U.S. Application Data
(63) Continuation of application No. PCT/US2020/021317, filed on Mar. 6, 2020.
(60) Provisional application No. 62/815,863, filed on Mar. 8, 2019.

(51) Int. Cl. G01N 33/574

(52) U.S. Cl. CPC G01N 33/57492; G01N 2333/70596

(57) ABSTRACT

The present disclosure provides assays for the detection and/or quantification of membrane-associated proteins, e.g., circulating CD20 (cCD20), incorporating an extracellular vesicle-based calibrator comprising the membrane-associated tumor antigen as well as the use of such assays in the detection and treatment of hyperproliferative disorders.

### CROSS-REFERENCE TO RELATED APPLICATIONS

This application is a continuation of International Patent Application No. PCT/US2020/021317, filed Mar. 6, 2020, which claims priority to U.S. Provisional Patent Application Ser. No. 62/815,863, filed Mar. 8, 2019, the contents of which are incorporated by reference in their entirety.

### FIELD OF THE INVENTION

The present disclosure provides assays for the detection and/or quantification of membrane-associated proteins, e.g., circulating CD20 (cCD20), incorporating an extracellular vesicle-based calibrator comprising the membrane-associated proteins as well as the use of such assays in the detection and treatment of hyperproliferative disorders.

### BACKGROUND

B-lymphocyte antigen CD20 (also called human B-lymphocyte-restricted differentiation antigen, Bp35) is a hydrophobic transmembrane protein with a molecular weight of approximately 35 kD. CD20 is can be detected on the surface of pre- and mature-B-lymphocytes. CD20 regulates an early step(s) in the activation process for B cell cycle initiation, cell differentiation, and cell proliferation. CD20 is also known to function as a calcium ion channel.

CD20 is a membrane protein with four transmembrane spanning regions that form a big extracellular loop on the cell surface and both N and C-termini located in the cytoplasm. Since both its C- and N-termini are located in the cytoplasm, on binding antibody, CD20 has been considered unlikely to be shed or cleaved from the cell surface. CD20 can form dimers or oligomers when translocating to lipid rafts. Given the expression of CD20 in B cell lymphomas, this antigen, like other membrane-associated tumor antigens, can serve as a candidate for “targeting” of such lymphomas. For example, antibodies specific to the CD20 surface antigen of B cells that bind to the extracellular loop have been administered to patients to facilitate the destruction and depletion of neoplastic B cells. Additionally, chemical agents or radioactive labels having the potential to destroy the tumor can be conjugated to anti-CD20 antibody such that the agent is specifically “delivered” to the neoplastic B cells. In view of the foregoing, CD20 antibodies are playing an increasing role in the therapy of patients with lymphoproliferative disorders, including those with chronic lymphocytic leukemia, non-Hodgkin's lymphoma, or Hodgkin's disease.

Membrane-associated proteins can circulate with other proteins in cell membrane fragments or large membrane complexes. For example, circulating CD20 protein can be present in the context of a membrane associated particle in circulation as a full-length protein. Accordingly, when present in circulation, these membrane-associated protein drug targets, such as CD20, can bind and sequester therapeutic antibodies and therefore, act as a drug sink for anti-CD20 antibodies, intended to target a tumor. Such sequestration can decrease the therapeutic efficiency of the therapeutic antibody. Membrane-associated proteins, e.g., circulating CD20, can also act as biomarkers for lymphoproliferative disorders such as chronic lymphocytic leukemia, non-Hodgkin's lymphoma, or Hodgkin's disease, as well as markers associated with the likelihood of a response to a treatment dependent on the presence of the specific tumor antigen. Given the significant role of membrane-associated proteins, e.g., circulating CD20, with respect to hyperproliferative disorder detection and treatment, there remains a need in the art for assays for determining the amount of membrane associated tumor antigen, e.g., circulating CD20, present in an individual.

### SUMMARY

The presently disclosed subject matter relates to assays and methods for the detection and/or quantification of a membrane protein, e.g., circulating CD20 (cCD20), incorporating an extracellular vesicle-based calibrator comprising the membrane-associated protein as well as the use of such assays in the detection and treatment of hyperproliferative disorders.

In certain embodiments, the present disclosure directed to assays for detecting a membrane-associated protein in a sample comprising: a) a capture antibody that binds to an extracellular vesicle comprising the membrane-associated protein in the sample thereby generating a capture antibody-extracellular vesicle complex, and b) a detection antibody that binds to the capture antibody-extracellular vesicle complex to form a detectable bound complex, wherein the signal from the detectable bound complex is calibrated against one or more known values detected from extracellular vesicle comprising the protein.

In certain embodiments, the assays of the present disclosure further comprise an extracellular vesicle calibrator.

In certain embodiments, the assays of the present disclosure comprise a capture antibody which does not compete for binding with a detection antibody. In certain embodiments, the capture antibody binds a different epitope than the detection antibody. In certain embodiments, the capture antibody is selected from the group consisting of: rituximab, ocrelizumab, ofatumumab, obinutuzumab, and combinations thereof. In certain embodiments, the detection antibody is selected from the group consisting of: rituximab, ocrelizumab, ofatumumab, obinutuzumab, and combinations thereof.

In certain embodiments, the present disclosure is directed to assays where a membrane-associated protein in a sample is utilized. In certain embodiments, the sample is selected from the group consisting of a plasma sample, a serum sample, a tissue culture supernatant sample, and combinations thereof. In certain embodiments, the membrane associated protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

In certain embodiments, the present disclosure is directed to methods for quantifying the concentration of circulating protein in a sample comprising the steps of: a) determining the level of a target protein in extracellular vesicles in the sample, and b) comparing the level of the protein in the extracellular vesicles in the sample with a calibration curve generated using extracellular vesicles comprising the target protein.

In certain embodiments, the present disclosure is directed to methods for quantifying the concentration of circulating protein in a sample comprising the steps of: a) generating a calibration curve using extracellular vesicles comprising the protein, and b) comparing the level of the target protein in extracellular vesicles in the sample with the calibration curve to determine the quantity of the protein in the extracellular vesicles in the sample.

In certain embodiments, the present disclosure is directed to methods for determining whether a patient with a B-cell lymphoma is likely to exhibit a response to an anti-CD20 therapy, comprising the steps of: a) obtaining a sample from the patient, b) determining the quantity of circulating CD20 in extracellular vesicles in the sample, c) comparing the level of CD20 in the extracellular vesicles in the sample with a calibration curve generated using extracellular vesicles comprising CD20, and d) determining whether the patient is likely to exhibit a response to the CD20 therapy, based on the quantity of circulating CD20 in extracellular vesicles determined in the sample. In certain embodiments, the anti-CD20 therapy comprises administration of an anti-CD20 antibody.

In certain embodiments, the present disclosure is directed to methods for determining the affinity of an anti-target protein antibody, e.g., an anti-CD20 antibody, comprising, subjecting the antibody to a surface plasmon resonance (SPR) analysis, wherein the SPR analysis comprises use of extracellular vesicles expressing the target protein, e.g., CD20, as a ligand and the antibody, e.g., anti-CD20 antibody, as an analyte. In certain embodiments, SPR analysis is employed as described herein to distinguish between two or more anti-target antibodies. In certain embodiments, SPR analysis allows for ranking of anti-target antibodies. In certain embodiments, selection of a particular anti-target antibody is performed by ranking two or more anti-target antibodies via SPR analysis and selecting the highest ranked anti-target antibody, or by selecting the anti-target antibody that exhibits a desired affinity.

In certain embodiments, the present disclosure is directed to methods for determining the activation of T cells obtained from a patient comprising a) incubating extracellular vesicles expressing CD20 with T cells and a CD20 T-cell dependent bispecific antibody, and b) determining the activation of T cells.

In certain embodiments, the present disclosure is directed to methods of treating a tumor in a subject in need comprising: a) obtaining a sample from the subject, b) generating a calibration curve using extracellular vesicles comprising a tumor antigen, c) comparing a level of the tumor antigen in extracellular vesicles in the sample with the calibration curve to determine the quantity of the target tumor antigen in the extracellular vesicles in the sample, d) determining whether the subject is likely to exhibit a response to an antibody therapy, based on the level of the tumor antigen in extracellular vesicles in the sample, and e) administering a therapeutic in response to the determination in d).

In certain embodiments, the methods of the present disclosure further comprising detecting the presence of extracellular vesicles using an extracellular marker, wherein the extracellular marker is selected from the group consisting of CD81, CD63, CD9, and combination thereof.

In certain embodiments, the present disclosure is directed to methods where the concentration of membrane associated protein and calibration curve are determined using an immunoassay, an ELISA, and/or a Western Blot. In certain embodiments, the sample is selected from the group consisting of a plasma sample, a serum sample, a tissue culture supernatant sample, and combinations thereof. In certain embodiments, the tumor antigen is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, and a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

In certain embodiments, the present disclosure is directed to methods utilizing an anti-CD 20 antibody, wherein the anti-CD20 antibody is selected from the group consisting of: rituximab, ocrelizumab, ofatumumab, obinutuzumab, a CD20 T-Cell dependent bispecific antibody, and combinations thereof.

### BRIEF DESCRIPTION OF THE FIGURES

FIG. 1A depicts an exemplary cCD20 structure which is present as a membrane associated particles in circulation as a full-length protein. FIG. 1B depicts exemplary intra-tetramer binding mechanism of Type I CD20 antibody.

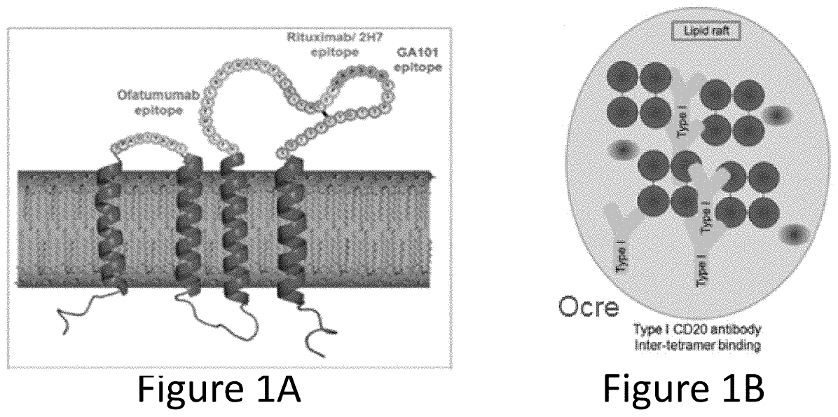

FIG. 2 depicts exemplary characterization of membrane associated vesicle calibrator by western blot, where column 1 represents a marker, column 2 represents EV 2 ug, column 3 represents EV 1 ug, column 4 represents EV 0.5 ug, column 5 represents EV 0.25 ug, column 6 represents rhCD20 250 ng, column 7 represents rCD20 100 ng, column 8 represents rCD20 40 ng, column 9 represents rCD20 16 ng, and column 10 represents rCD20 6.4 ng.

FIG. 3 depicts exemplary characterization of CD20 in membrane and/or extracellular vesicles.

FIG. 4 depicts the results of capture a detection with either (left panel) two anti-CD20 antibodies; or (right panel) capture with an anti-CD20 antibody and detection with anti-CD9, anti-CD63, and anti-CD81 antibodies.

FIG. 5A depicts an exemplary 3-day sequential assay format. FIG. 5B depicts an exemplary bridge assay format.

FIG. 6 depicts an exemplary alternative immunoassay format.

FIG. 7 depicts an exemplary analysis of the impact of detergent on CD20 detection.

FIG. 8 depicts an exemplary drug tolerance analysis.

FIG. 9A depicts an exemplary assay format for characterization of anti-CD20 TDB to CD20 expressed on extracellular vesicles. FIG. 9B depicts exemplary binding affinities of anti-CD20 TDB to CD20 EVs at determined by kinetic analysis and Biacore Sensorgram.

FIG. 10 depicts an exemplary standard curve.

### DETAILED DESCRIPTION

The present disclosure provides assays for the detection and/or quantification of membrane-associated proteins, e.g., circulating CD20, incorporating an extracellular vesicle-based calibrator comprising the membrane-associated proteins as well as the use of such assays in the detection and treatment of hyperproliferative disorders. In certain embodiments, the assays of the present disclosure comprise quantifying the concentration of membrane-associated, e.g., extracellular vesicle-associated, protein, e.g., circulating CD20, by determining the level of CD20 present in extracellular vesicles in a sample and comparing the level of CD20 in the sample with a calibration curve generated using extracellular vesicles comprising CD20. In certain embodiments, an immunoassay employing one or more antibodies, e.g., an ELISA or a western blot, is employed to determine the concentration of membrane-associated protein in a sample or in connection with the preparation of a calibration curve.

For clarity, but not by way of limitation, the detailed description of the presently disclosed subject matter is divided into the following subsections:

I. Definitions;

II. Immunoassays;

III. Antibodies;

IV. Kits; and

V. Exemplary Embodiments.

### I. DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as ±10% of the value modified by the term “about.”

The term “acceptor human framework” for the purposes herein refers a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In certain embodiments, the number of amino acid changes are 10 or less, 9 or less, 8 or less, 7 or less, 6 or less, 5 or less, 4 or less, 3 or less, or 2 or less. In certain embodiments, the VL acceptor human framework is identical in sequence to the VL human immunoglobulin framework sequence or human consensus framework sequence.

The term “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

The term “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody which binds” an antigen of interest, e.g., a CD20 protein, is one that binds the antigen with sufficient affinity such that the antibody is useful as an assay reagent, e.g., as a capture antibody or as a detection antibody. Typically, such an antibody does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a target molecule compared to binding of a control molecule, which generally is a molecule of similar structure that does not have binding activity.

The terms “anti-tumor antigen antibody” refers to an antibody that is capable of binding a tumor antigen, e.g., CD20, with sufficient affinity such that the antibody is useful as an agent in targeting the tumor antigen, e.g., as an agent in the assays described herein. In certain embodiments, the extent of binding of an anti-tumor antigen antibody to an unrelated protein is less than about 10% of the binding of the antibody to the targeted tumor antigen as measured, e.g., by a radioimmunoassay (RIA). In certain embodiments, an antibody that binds to the targeted tumor antigen has a dissociation constant (K_{D}) of ≤1 M, ≤100 mM, ≤10 mM, ≤1 mM, ≤100 μM, ≤10 μM, ≤1 μM, ≤100 nM, ≤10 nM, ≤1 nM, ≤0.1 nM, ≤0.01 nM or ≤0.001 nM. In certain embodiments, the K_{D} of an antibody that binds to CD20, disclosed herein, can be 10⁻³M or less or 10⁻¹⁰ M or less, e.g., from 10⁻⁸ M to 10⁻¹³ M, e.g., from 10⁻⁹ M to 10⁻¹³M. In certain embodiments, the K_{D} of an antibody that binds to the targeted tumor antigen, disclosed herein, can be 10⁻¹⁰ M to 10⁻¹³ M. In certain embodiments, an anti-tumor antigen antibody binds to an epitope of the targeted tumor antigen that is conserved among the targeted tumor antigen from different species.

An “antibody that competes for binding” with a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is described in “Antibodies,” Harlow and Lane (Cold Spring Harbor Press, Cold Spring Harbor, N.Y.).

A “B-cell” is a lymphocyte that matures within the bone marrow, and includes a naive B cell, memory B cell, or effector B cell (plasma cells). The B-cell herein may be a normal or non-malignant B-cell.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

A “capture antibody,” as used herein, refers to an antibody that specifically binds a target molecule, e.g., a form of CD20, in a sample. Under certain conditions, the capture antibody forms a complex with the target molecule such that the antibody-target molecule complex can be separated from the rest of the sample. In certain embodiments, such separation may include washing away substances or material in the sample that did not bind the capture antibody. In certain embodiments, a capture antibody may be attached to a solid support surface, such as, for example but not limited to, a plate or a bead, e.g., a paramagnetic bead.

The term “CD20”, as used herein, refers to CD20 antigen which is an approximate 35 kDa, phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35”. The CD20 antigen is described in Clark et al. PNAS (USA) 82:1766 (1985), for example.

The term “CD20 nucleic acid” herein refers to nucleic acid, including DNA and mRNA, that encodes at least a portion of the CD20 protein, and/or the complementary nucleic acid.

By “detecting tumor antigen” is meant evaluating whether a sample comprises the tumor antigen. Generally, the tumor antigen protein, e.g., CD20 protein, will be detected, but detecting tumor antigen nucleic acid, e.g., CD20 nucleic acid, is also encompassed by this phrase herein.

The term “tumor antigen nucleic acid” herein refers to nucleic acid, including DNA and mRNA, that encodes at least a portion of the tumor antigen protein, and/or the complementary nucleic acid.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

Throughout this specification and claims, the word “comprise,” or variations such as “comprises” or “comprising,” will be understood to imply the inclusion of a stated integer or group of integers but not the exclusion of any other integer or group of integers.

The terms “correlate” or “correlating” refer to the comparison, in any way, of the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

The term “detecting,” is used herein, to include both qualitative and quantitative measurements of a target molecule, e.g., CD20 or processed forms thereof. In certain embodiments, detecting includes identifying the mere presence of the target molecule in a sample as well as determining whether the target molecule is present in the sample at detectable levels.

The term “detection antibody,” as used herein, refers to an antibody that specifically binds a target molecule in a sample or in a sample-capture antibody combination material. Under certain conditions, the detection antibody forms a complex with the target molecule or with a target molecule-capture antibody complex. A detection antibody is capable of being detected either directly through a label, which may be amplified, or indirectly, e.g., through use of another antibody that is labeled and that binds the detection antibody. For direct labeling, the detection antibody is typically conjugated to a moiety that is detectable by some means, for example, including but not limited to, biotin or ruthenium.

The term “detection means,” as used herein, refers to a moiety or technique used to detect the presence of the detectable antibody through signal reporting that is then read out in an assay. Typically, a detection means employ reagents, e.g., a detection agent, that amplify an immobilized label such as the label captured onto a microtiter plate, e.g., avidin, streptavidin-HRP or streptavidin-β-D-galactopyranose.

An “effective amount” of an agent refers to the amount that is necessary to result in a physiological change in the cell or tissue to which it is administered.

The term “Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody” and “whole antibody” are used herein interchangeably to refer to an antibody having a structure substantially similar to a native antibody structure or having heavy chains that contain an Fc region as defined herein.

A “heteromultimer”, “heteromultimeric complex”, or “heteromultimeric protein” refers to a molecule comprising at least a first hinge-containing polypeptide and a second hinge-containing polypeptide, wherein the second hinge-containing polypeptide differs in amino acid sequence from the first hinge-containing polypeptide by at least one amino acid residue. The heteromultimer can comprise a “heterodimer” formed by the first and second hinge-containing polypeptides or can form higher order tertiary structures where polypeptides in addition to the first and second hinge-containing polypeptides are present. The polypeptides of the heteromultimer may interact with each other by a non-peptidic, covalent bond (e.g., disulfide bond) and/or a non-covalent interaction (e.g., hydrogen bonds, ionic bonds, van der Waals forces, and/or hydrophobic interactions).

The terms “host cell,” “host cell line,” and “host cell culture” as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., HVRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein, refers to each of the regions of an antibody variable domain which are hypervariable in sequence (also referred to herein as “complementarity determining regions” or “HVRs”) and/or form structurally defined loops (“hypervariable loops”) and/or contain the antigen-contacting residues (“antigen contacts”). Unless otherwise indicated, HVR residues and other residues in the variable domain (e.g., FR residues) are numbered herein according to Kabat et al., supra. Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) HVRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3). An “individual” or “subject,” as used interchangeably herein, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “immunoconjugate” refers to an antibody conjugated to one or more heterologous molecule(s), including but not limited to a cytotoxic agent.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “individual” or “subject,” as used interchangeably herein, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

“Isolated nucleic acid encoding an antibody” (including references to a specific antibody, e.g., an anti-CD20 antibody) refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “label” or “detectable label,” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label may signal its presence by participating in specific binding reactions. Non-limiting examples of such labels include haptens, antibodies, biotin, streptavidin, his-tag, nitrilotriacetic acid, glutathione S-transferase, glutathione and the like.

The term “membrane-associated protein”, as used here in, refers to any membrane associated targets including antigens, peptides, and proteins. The membrane-associated proteins can include integral membrane proteins and/or peripheral membrane proteins. Non-limiting examples of membrane-associated proteins include, but are not limited to, a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, and a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert,” as used herein, refers to instructions customarily included in commercial packages that contain information concerning the use of the components of the package.

A “pathogenic” cell is one which causes a disease or abnormality, and may be present in or around diseased tissue or cells.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X \text{/} Y$$

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

A “sample,” as used herein, refers to a small portion of a larger quantity of material. In certain embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy or aspirate), blood or any blood constituents, bodily fluids (such as, e.g., urine, lymph, cerebral spinal fluid, amniotic fluid, peritoneal fluid or interstitial fluid), or cells from the individual, including circulating cells.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In certain embodiments, methods and compositions of the present disclosure are useful in attempts to delay development of a disease or disorder.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. IMMUNOASSAYS

The present disclosure provides assays for the detection and/or quantification of membrane-associated proteins, e.g., circulating CD20, incorporating an extracellular vesicle-based calibrator comprising the membrane-associated proteins, as well as the use of such assays in the detection and treatment of hyperproliferative disorders. In certain embodiments, the assays of the present disclosure comprise quantifying the concentration of membrane-associated, e.g., extracellular vesicle-associated, protein, e.g., circulating CD20, by determining the level of CD20 present in extracellular vesicles in a sample and comparing the level of CD20 in the sample with a calibration curve generated using extracellular vesicles comprising CD20. In certain embodiments, an immunoassay employing one or more antibodies, e.g., an ELISA or a western blot, is employed to determine the concentration of membrane-associated tumor antigen in a sample or in connection with the preparation of a calibration curve.

In certain embodiments, the present disclosure provides immunoassay methods for the detection and quantification of membrane-associated proteins. For example, the immunoassay methods of the present disclosure can incorporate strategies known in the art, including but not limited to, sandwich assay, enzyme-linked immunosorbent assay (ELISA) assay, a digital form of ELISA, electrochemical assay (ECL) assay and magnetic immunoassay.

In certain embodiments, the present disclosure provides an extracellular vesicle (EV)-based calibrator. The EV calibrator can be a membrane bound protein calibrator. For example, the EV calibrator can be similar in confirmation to native CD20. Since ocrelizumab binds to an epitope of CD20 that has tertiary structure (in the form of a loop) resulting from four transmembrane spans, it is important to generate a membrane bound protein calibrator.

In certain embodiments, the present disclosure provides methods for generating the EV calibrator. An exemplary method includes passaging a seed train every 3 to 4 days, dilute the seed train in a production media to a production culture, transfecting the production culture (e.g., DNA/jetPEI complex), collecting the EV calibrator from the transfected production culture, and purifying the EV calibrator. The purified EV calibrator can be characterized through western lot.

In certain embodiments, the present disclosure provides methods for a sequential assay. The sequential can be a three-day sequential assay. The three-day sequential assay can be used in instances where improved sensitivity is desirable. For example, on the first day, a plate can be coated with capture antibodies at 4° C. On the second day, samples can be added to the plate and incubated overnight at 4° C. On the third day, detection antibody (e.g., conjugated to biotin) and reagents (e.g., HRP). In non-limiting embodiments, Ocre (capture antibody), Ofa (detection antibody) and HRP 100 ng/mL (signal) can be used for the three-day sequential assay.

In certain embodiments, the present disclosure provides methods for a bridging assay. The bridging assays can be a two-day bridging assay. The two-day bridge assay can be used in instances where reduced time to results is desirable. For example, on the first day, a sample can be incubated with a master mix which includes biotin conjugated ocrelizumab and dig conjugated ofatumumab antibodies. On the second day, sample can be transferred to a streptavidin plate and subsequently, HRP conjugated anti-DIG antibody can be added and incubated for detection. In non-limiting embodiments, the plates can be read at 450 nm for detection absorbance and 630 nm for reference absorbance. Sample concentration can be determined by entering data into a five-parameter logistic curve-fitting with 1/y² curve weighting.

In certain embodiments, the methods of the present disclosure comprise contacting a sample obtained from a subject with a capture antibody, such as those described herein, under conditions permissive for binding of the capture anti-CD20 antibody to CD20 protein in the sample. For example, but not by way of limitation, the sample can be incubated with a capture antibody that binds to an epitope present on CD20 to generate a sample-capture antibody combination material. The conditions for the incubation of the sample and the capture antibody can be selected to maximize the sensitivity of the assay and/or to minimize dissociation, as well as to ensure that the CD20 protein present in the sample binds to the capture antibody.

In certain embodiments, the capture antibodies used in the immunoassay methods disclosed herein can be used at a concentration from about 0.1 μg/ml to about 5.0 μg/ml. For example, but not by way of limitation, the capture antibodies can be used at a concentration from about 0.1 μg/ml to about 0.5 μg/ml, from about 0.1 μg/ml to about 1.0 μg/ml, from about 0.1 μg/ml to about 1.5 μg/ml, from about 0.1 μg/ml to about 2.0 μg/ml, from about 0.1 μg/ml to about 2.5 μg/ml, from about 0.1 μg/ml to about 3.0 μg/ml, from about 0.1 μg/ml to about 3.5 μg/ml, from about 0.1 μg/ml to about 4.0 μg/ml, from about 0.1 μg/ml to about 4.5 μg/ml, from about 0.5 μg/ml to about 5.0 μg/ml, from about 1.0 μg/ml to about 5.0 μg/ml, from about 1.5 μg/ml to about 5.0 μg/ml, from about 2.0 μg/ml to about 5.0 μg/ml, from about 2.5 μg/ml to about 5.0 μg/ml, from about 3.0 μg/ml to about 5.0 μg/ml, from about 3.5 μg/ml to about 5.0 μg/ml, from about 4.0 μg/ml to about 5.0 μg/ml, from about 4.5 μg/ml to about 5.0 μg/ml, from about 0.5 μg/ml to about 2.0 μg/ml or from about 0.5 μg/ml to about 1.0 μg/ml, e.g., about 0.5 μg/ml.

In certain embodiments, the capture antibody can be diluted in a coating buffer. Non-limiting examples of coating buffers include PBS, a carbonate buffer, a bicarbonate buffer or combinations thereof. In certain embodiments, the coating buffer is sodium bicarbonate. In certain embodiments, the coating buffer is PBS. In certain embodiments, the coating buffer can be used at a concentration from about 10 mM to about 1 M. For example, but not by way of limitation, the coating buffer can be used at a concentration from about 10 mM to about 100 mM, from about 10 mM to about 200 mM, from about 10 mM to about 300 mM, from about 10 mM to about 400 mM, from about 10 mM to about 500 mM, from about 10 mM to about 600 mM, from about 10 mM to about 700 mM, from about 10 mM to about 800 mM, from about 10 mM to about 900 mM, from about 100 mM to about 1 M, from about 200 mM to about 1 M, from about 300 mM to about 1 M, from about 400 mM to about 1 M, from about 500 mM to about 1 M, from about 600 mM to about 1 M, from about 700 mM to about 1 M, from about 800 mM to about 1 M or from about 900 mM to about 1 M.

Capture antibodies, as used herein, can be immobilized on a solid phase. For example, but not by way of limitation, the solid phase can be any inert support or carrier that is useful in immunometric assays, including supports in the form of, e.g., surfaces, particles, porous matrices, beads and the like. Non-limiting examples of commonly used supports include small sheets, SEPHADEX®, gels, polyvinyl chloride, plastic beads and assay plates or test tubes manufactured from polyethylene, polypropylene, polystyrene, and the like, including 96-well microtiter plates, as well as particulate materials such as filter paper, agarose, cross-linked dextran, and other polysaccharides. In certain embodiments, the solid phase used for immobilization can be beads. For example, but not by way of limitation, a capture antibody disclosed herein is immobilized to paramagnetic beads. In certain embodiments, the immobilized capture antibodies are coated on a microtiter plate that can be used to analyze several samples at one time.

In certain embodiments, the paramagnetic beads coupled to the capture antibody can be used at a concentration from about 0.1×10⁷ beads/ml to about 10.0×10⁷ beads/ml, e.g., from about 0.1×10⁷ beads/ml to about 0.5×10⁷ beads/ml, from about 0.1×10⁷ beads/ml to about 1.0×10⁷ beads/ml, from about 0.1×10⁷ beads/ml to about 2.0×10⁷ beads/ml, from about 0.1×10⁷ beads/ml to about 3.0×10⁷ beads/ml, from about 0.1×10⁷ beads/ml to about 4.0×10⁷ beads/ml, from about 0.1×10⁷ beads/ml to about 5.0×10⁷ beads/ml, from about 0.1×10⁷ beads/ml to about 6.0×10⁷ beads/ml, from about 0.1×10⁷ beads/ml to about 7.0×10⁷ beads/ml, from about 0.1×10⁷ beads/ml to about 8.0×10⁷ beads/ml, from about 0.1×10⁷ beads/ml to about 9.0×10⁷ beads/ml, from about 0.5×10⁷ beads/ml to about 10.0×10⁷ beads/ml, from about 1.0×10⁷ beads/ml to about 10.0×10⁷ beads/ml, from about 2.0×10⁷ beads/ml to about 10.0×10⁷ beads/ml, from about 3.0×10⁷ beads/ml to about 10.0×10⁷ beads/ml, from about 4.0×10⁷ beads/ml to about 10.0×10⁷ beads/ml, from about 5.0×10⁷ beads/ml to about 10.0×10⁷ beads/ml, from about 6.0×10⁷ beads/ml to about 10.0×10⁷ beads/ml, from about 7.0×10⁷ beads/ml to about 10.0×10⁷ beads/ml, from about 8.0×10⁷ beads/ml to about 10.0×10⁷ beads/ml, from about 9.0×10⁷ beads/ml to about 10.0×10⁷ beads/ml, from about 0.5×10⁷ beads/ml to about 1.0×10⁷ beads/ml, from about 0.5×10⁷ beads/ml to about 2.0×10⁷ beads/ml or from about 0.5×10⁷ beads/ml to about 3.0×10⁷ beads/ml. In certain embodiments, the paramagnetic beds can be used at a concentration from about 0.5×10⁷ beads/ml to about 2.0×10⁷ beads/ml. In certain embodiments, the paramagnetic beds can be used at a concentration of about 1.0×10⁷ beads/ml, e.g., about 1.22×10⁷ beads/ml, or at a concentration of about 0.5×10⁷ beads/ml, e.g., about 0.59×10⁷ beads/ml.

The immunoassay methods disclosed herein can further include contacting a sample-capture antibody combination material with a detector antibody. In certain embodiments, the detector antibody binds to an epitope present on CD20. In certain embodiments, the detector antibody binds to an epitope present on the sample-capture antibody combination material, but not on the capture antibody in the absence of CD20. In certain embodiments, the detector antibody bound to the sample-capture antibody combination is subsequently measured or quantified using a detection means, e.g., one or more detection agents, for the detection antibody to determine the amount of CD20 protein bound by the detector antibody.

In certain embodiments, the detector antibody can be used in a concentration from about 0.1 μg/ml to about 5.0 μg/ml. For example, but not by way of limitation, the detector antibody can be used at a concentration from about 0.1 μg/ml to about 0.5 μg/ml, from about 0.1 μg/ml to about 1.0 μg/ml, from about 0.1 μg/ml to about 1.5 μg/ml, from about 0.1 μg/ml to about 2.0 μg/ml, from about 0.1 μg/ml to about 2.5 μg/ml, from about 0.1 μg/ml to about 3.0 μg/ml, from about 0.1 μg/ml to about 3.5 μg/ml, from about 0.1 μg/ml to about 4.0 μg/ml, from about 0.1 μg/ml to about 4.5 μg/ml, from about 0.5 μg/ml to about 5.0 μg/ml, from about 1.0 μg/ml to about 5.0 μg/ml, from about 1.5 μg/ml to about 5.0 μg/ml, from about 2.0 μg/ml to about 5.0 μg/ml, from about 2.5 μg/ml to about 5.0 μg/ml, from about 3.0 μg/ml to about 5.0 μg/ml, from about 3.5 μg/ml to about 5.0 μg/ml, from about 4.0 μg/ml to about 5.0 μg/ml, from about 4.5 μg/ml to about 5.0 μg/ml, from about 1.0 μg/ml to about 3.0 μg/ml, from about 0.5 μg/ml to about 3.0 μg/ml or from about 0.5 μg/ml to about 2.0 μg/ml. In certain embodiments, an immunoassay for detecting total CD20 protein can use a detector antibody at a concentration between about 0.1 μg/ml to about 1.0 μg/ml, e.g., about 0.4 μg/ml or about 0.8 μg/ml. In certain embodiments, an immunoassay for detecting active CD20 protein can use a detector antibody at a concentration between about 1.0 μg/ml to about 3.0 μg/ml, e.g., about 1.1 μg/ml or about 2.1 μg/ml.

In certain embodiments, the anti-CD20 antibodies for use in the disclosed methods can be labeled. Labels include, but are not limited to, labels or moieties that are detected directly, such as fluorescent, chromophoric, electron-dense, chemiluminescent, and radioactive labels, as well as moieties, such as enzymes or ligands, that are detected indirectly, e.g., through an enzymatic reaction or molecular interaction. Non-limiting examples of labels include the radioisotopes ³²P, ¹⁴C, ¹²⁵I, ³H and ¹³¹I, fluorophores such as rare earth chelates or fluorescein and its derivatives, rhodamine and its derivatives, dansyl, umbelliferone, luceriferases, e.g., firefly luciferase and bacterial luciferase (see U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase or microperoxidase, biotin/avidin, spin labels, bacteriophage labels, stable free radicals and the like. In certain embodiments, the detector antibody is labeled with biotin, e.g., the detector antibody is conjugated to biotin.

In certain embodiments, the detection agent for the biotinylated detector antibody is avidin, streptavidin-HRP or streptavidin-β-D-galactopyranose (SBG). In certain embodiments, the readout of the detection agent is fluorimetric or colorimetric. For example, but not by way of limitation, tetramethylbenzidine and hydrogen peroxide can be used as the readout. In certain embodiments, if the detection agent is streptavidin-HRP, the readout can be colorimetric by using tetramethylbenzidine and hydrogen peroxide. Alternatively, in certain embodiments, resorufin β-D-galactopyranoside can be used as the readout. For example, but not by way of limitation, if the detection agent is SBG, the readout can be fluorimetric by using resorufin β-D-galactopyranoside.

In certain embodiments, the detection agent, e.g., SBG, can be used at a concentration from about 50 to about 500 pM. For example, but not by way of limitation, the detection agent can be used at a concentration from about 50 to about 100 pM, from about 50 to about 150 pM, from about 50 to about 200 pM, from about 50 to about 250 pM, from about 50 to about 300 pM, from about 50 to about 350 pM, from about 50 to about 400 pM, from about 50 to about 450 pM, from about 100 to about 500 pM, from about 150 to about 500 pM, from about 200 to about 500 pM, from about 250 to about 500 pM, from about 300 to about 500 pM, from about 350 to about 500 pM, from about 400 to about 500 pM, from about 450 to about 500 pM, from about 100 to about 400 pM or from about 200 to about 400 pM. In certain embodiments, the detection agent can be used at a concentration from about 100 pM to about 400 pM, e.g., SBG can be used at a concentration of about 110 pM, about 155 pM or about 310 pM. In certain embodiments, SBG is used at a concentration of about 310 pM. In certain embodiments, the detection agent, e.g., HRP, can be used at a dilution from about 1/10 to about 1/1000. For example, but not by way of limitation, the detection agent can be used at a dilution from about 1/10 to about 1/100, from about 1/10 to about 1/500, from about 1/100 to about 1/1000 or from about 1/500 to about 1/1000. In certain embodiments, the detection agent can be used at a dilution from about 1/100 to about 1/1000, e.g., HRP can be used at a dilution of about 1/100 or about 1/500.

In certain embodiments, the methods of the present disclosure can include blocking the capture antibody with a blocking buffer. In certain embodiments, the blocking buffer can include PBS, bovine serum albumin (BSA) and/or a biocide, e.g., ProClin™ (Sigma-Aldrich, Saint Louis, Mo.). In certain embodiments, the method can include multiple washing steps. In certain embodiments, the solution used for washing is generally a buffer (e.g., a “washing buffer”) such as, but not limited to, a PBS buffer that includes a detergent, e.g., Tween 20. For example, but not by way of limitation, the capture antibody can be washed after blocking and/or the sample can be separated from the capture antibody to remove uncaptured material, e.g., by washing.

In certain embodiments, the immunoassay methods disclosed herein have a detection sensitivity, e.g., an in-well sensitivity, from about 2 pg/ml to about 20 pg/ml. For example, but not by way of limitation, an immunoassay disclosed herein has a sensitivity from about 2 pg/ml to about 3 pg/ml, from about 2 pg/ml to about 4 pg/ml, from about 2 pg/ml to about 5 pg/ml, from about 2 pg/ml to about 6 pg/ml, from about 2 pg/ml to about 7 pg/ml, from about 2 pg/ml to about 8 pg/ml, from about 2 pg/ml to about 10 pg/ml, from about 2 pg/ml to about 11 pg/ml, from about 2 pg/ml to about 12 pg/ml, from about 2 pg/ml to about 13 pg/ml, from about 2 pg/ml to about 14 pg/ml, from about 2 pg/ml to about 15 pg/ml, from about 2 pg/ml to about 16 pg/ml, from about 2 pg/ml to about 17 pg/ml, from about 2 pg/ml to about 18 pg/ml, from about 2 pg/ml to about 19 pg/ml, from about 3 pg/ml to about 15 pg/ml, from about 3 pg/ml to about 10 pg/ml or from about 3 pg/ml to about 5 pg/ml. In certain embodiments, an immunoassay disclosed herein has a sensitivity of about 2 pg/ml or greater, 1 pg/ml or greater or 0.5 pg/ml or greater. In certain embodiments, an immunoassay disclosed herein has a detection sensitivity, e.g., an in-well sensitivity, from about 0.2 pg/ml to about 2.0 pg/ml, e.g., from about 0.2 pg/ml to about 0.5 pg/ml, from about 0.2 pg/ml to about 1.0 pg/ml or from about 0.2 pg/ml to about 1.5 pg/ml. For example, but not by way of limitation, an immunoassay disclosed herein, e.g., a single molecule immunoassay using the Simoa HD-1 Analyzer™ has a sensitivity, e.g., an in-well sensitivity, from about 0.2 pg/ml to about 0.5 pg/ml.

The samples analyzed by the immunoassay methods of the present disclosure can be clinical samples, cells in culture, cell supernatants, cell lysates, serum samples, blood plasma samples, other biological fluid (e.g., lymphatic fluid) samples or tissue samples. In certain embodiments, the source of the sample may be solid tissue (e.g., from a fresh, frozen and/or preserved organ, tissue sample, serum, blood plasma, biopsy or aspirate) or cells from a subject. In certain embodiments, the sample is a blood sample. In certain embodiments, the sample is a plasma sample. In certain embodiments, the sample, e.g., blood or plasma sample, can be obtained from a subject and treated with one or more protease, esterase, DDP-IV and/or phosphatase inhibitors. For example, but not by way of limitation, a sample can be treated with a cocktail of protease and phosphatase inhibitors, e.g., MS-SAFE (Sigma-Aldrich, Saint Louis, Mo.). In certain embodiments, the sample is treated with an anti-coagulant or collected in tube that contains an anti-coagulant, e.g., K₂-EDTA. In certain embodiments, the sample can be collected using the P800 Blood Collection System (BD Biosciences, San Jose, Calif.).

In certain embodiments, the present disclosure provides methods for measuring the affinity of therapeutic agents using surface plasmon resonance analysis (SPR). For example, binding interactions between a target protein, e.g., CD20, expressed on extracellular vesicles and an anti-target protein antibody, e.g., an anti-CD20 antibody, can be evaluated by the SPR analysis, where the extracellular vesicles expressing the target, e.g., CD20, can be used as a ligand and the anti-target antibody, e.g., anti-CD20 antibody, can be used as an analyte. Through the SPR analysis, the dissociation equilibrium constant (K_{D}), dissociation rate constant (k_{d}), and association rate constant (kₐ) values can be calculated. In certain embodiments, the therapeutic agents can include rituximab, ocrelizumab, ofatumumab, obinutuzumab, a CD20 T-Cell dependent bispecific antibody, and combinations thereof. In certain embodiments, SPR analysis is employed as described herein to distinguish between two or more anti-target antibodies. In certain embodiments, SPR analysis allows for ranking of anti-target antibodies. In certain embodiments, selection of a particular anti-target antibody is performed by ranking two or more anti-target antibodies via SPR analysis and selecting the highest ranked anti-target antibody, or by selecting the anti-target antibody that exhibits a desired affinity.

### III. ANTIBODIES

The present disclosure further provides antibodies that bind to CD20. Antibodies of the present disclosure are useful for detecting and quantifying CD20 protein levels in a sample. In certain embodiments, the antibodies of the present disclosure can be used in immunoassay methods for the detection and quantification of CD20 protein, disclosed herein. For example, but not by way of limitation, antibodies of the present disclosure can be used to detect the levels of circulating CD20 protein in a sample.

In certain embodiments, an antibody of the present disclosure can be humanized. In certain embodiments, an antibody of the present disclosure comprises an acceptor human framework, e.g., a human immunoglobulin framework or a human consensus framework. In certain embodiments, an antibody of the present disclosure can be a monoclonal antibody, including a chimeric, humanized or human antibody. In certain embodiments, an antibody of the present disclosure can be an antibody fragment, e.g., a Fv, Fab, Fab′, scFv, diabody or F(ab′)₂ fragment. In certain embodiments, the antibody is an IgG. In certain embodiments, the antibody is selected from IgG1, IgG2, IgG3 and IgG4. In certain embodiments, the antibody is a full-length antibody, e.g., an intact IgG1 antibody, or other antibody class or isotype as defined herein. In certain embodiments, the antibodies disclosed herein can be labeled, e.g., conjugated to biotin. In certain embodiments, an antibody of the present disclosure can incorporate any of the features, singly or in combination, as described in Sections 1-7, detailed below.

A. Exemplary Antibodies

In certain embodiments, the antibodies, e.g., anti-CD20 antibodies, of the present disclosure can have a dissociation constant (K_{D}) of ≤1 M, ≤100 mM, ≤10 mM, ≤1 mM, ≤100 μM, ≤10 μM, ≤1 μM, ≤100 nM, ≤10 nM, ≤1 nM, ≤0.1 nM, ≤0.01 nM or ≤0.001 nM. In certain embodiments, an antibody of the present disclosure can have a K_{D} of about 10⁻³ or less or 10⁻⁸ M or less, e.g., from 10⁻¹³ M to 10⁻¹³ M, e.g., from 10⁻⁹ M to 10⁻¹³ M. In certain embodiments, an antibody, disclosed herein, can have a K_{D} of about 10⁻¹⁰ M to 10⁻¹³ M. For example, but not by way of limitation, a capture antibody or a detector antibody of the present disclosure binds to its target antigen with a K_{D} from about 10⁻¹⁰ M to 10⁻¹³ M.

In certain embodiments, K_{D} can be measured by a radiolabeled antigen binding assay (RIA). In certain embodiments, an RIA can be performed with a Fab version of an antibody of interest and its antigen. For example, but not by way of limitation, a solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

In certain embodiments, K_{D} can be measured using a BIACORE® surface plasmon resonance assay. For example, but not by way of limitation, an assay using a BIACORE®-2000, a BIACORE®-3000, a BIACORE X100 or a BIACORE T200 processing unit (Biacore, Inc., Piscataway, N.J.) is performed at 25° C. with immobilized antigen CMS chips at ˜10 response units (RU). In certain embodiments, carboxymethylated dextran biosensor chips (CMS, Biacore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) can be calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

1. Antibody Fragments

In certain embodiments, an antigen antibody of the present disclosure is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For a discussion of Fab and F(ab′)₂ fragments comprising salvage receptor binding epitope residues and having increased in vivo half-life, see U.S. Pat. No. 5,869,046.

In certain embodiments, an antibody of the present disclosure can be a diabody. Diabodies are antibody fragments comprising two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies, which are additional antibody fragments within the scope of the antibodies of the present disclosure, are also described in Hudson et al., Nat. Med. 9:129-134 (2003).

In certain embodiments, an antibody of the present disclosure can be a single-domain antibody. Single-domain antibodies are antibody fragments that comprise all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques including, but not limited to, proteolytic digestion of an intact antibody as well as production by recombinant host cells (e.g., E. coli or phage), as described herein.

2. Chimeric and Humanized Antibodies

In certain embodiments, an antibody of the present disclosure is a chimeric antibody. Certain chimeric antibodies are described in the art, e.g., in U.S. Pat. No. 4,816,567; and Morrison et al., Proc. Natl. Acad. Sci. USA, 81:6851-6855 (1984)). In certain embodiments, a chimeric antibody of the present disclosure comprises a non-human variable region (e.g., a variable region derived from a mouse, rat, hamster, rabbit or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In certain embodiments, a chimeric antibody of the present disclosure can be a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

3. Human Antibodies

In certain embodiments, an antibody of the present disclosure can be a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boemer et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

4. Library-Derived Antibodies

Antibodies of the present disclosure can be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). In certain embodiments, naive libraries can also be made synthetically by cloning unrearranged V-gene segments from stem cells, and using PCR primers containing random sequence to encode the highly variable HVR regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

5. Multispecific Antibodies

In certain embodiments, an antibody of the present disclosure can be a multispecific antibody, e.g., a bispecific antibody. Multispecific antibodies are monoclonal antibodies that have binding specificities for at least two different epitopes. In certain embodiments, one of the binding specificities is for an epitope present on CD20 and the other is for any other antigen. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

6. Antibody Variants

The presently disclosed subject matter further provides amino acid sequence variants of the disclosed antibodies. For example, it may be desirable to improve the binding affinity and/or other biological properties of the antibody. Amino acid sequence variants of an antibody can be prepared by introducing appropriate modifications into the nucleotide sequence encoding the antibody or by peptide synthesis. Such modifications include, but are not limited to, deletions from, and/or insertions into and/or substitutions of residues within the amino acid sequences of the antibody. Any combination of deletion, insertion, and substitution can be made to arrive at the final construct, provided that the final antibody, i.e., modified, possesses the desired characteristics, e.g., antigen-binding.

a) Substitution, Insertion, and Deletion Variants

Antibody variants can have one or more amino acid substitutions, insertions and/or deletions. Sites of interest for such variation include, but are not limited to, the HVRs, and FRs. Non-limiting examples of conservative substitutions are shown in Table 1 under the heading of “preferred substitutions.” Non-limiting examples of more substantial changes are provided in Table 1 under the heading of “exemplary substitutions,” and as further described below in reference to amino acid side chain classes. Amino acid substitutions can be introduced into an antibody of interest and the products screened for a desired activity, e.g., retained/improved antigen binding, decreased immunogenicity or improved complement dependent cytotoxicity (CDC) or antibody-dependent cell-mediated cytotoxicity (ADCC).

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

In certain embodiments, non-conservative substitutions will entail exchanging a member of one of these classes for another class.

In certain embodiments, a type of substitutional variant involves substituting one or more hypervariable region residues of a parent antibody, e.g., a humanized or human antibody. Generally, the resulting variant(s) selected for further study will have modifications, e.g., improvements, in certain biological properties such as, but not limited to, increased affinity, reduced immunogenicity, relative to the parent antibody and/or will have substantially retained certain biological properties of the parent antibody. A non-limiting example of a substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

In certain embodiments, alterations (e.g., substitutions) can be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In certain embodiments of affinity maturation, diversity can be introduced into the variable genes chosen for maturation by any of a variety of methods (e.g., error-prone PCR, chain shuffling or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding can be specifically identified, e.g., using alanine scanning mutagenesis or modeling.

In certain embodiments, substitutions, insertions and/or deletions can occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for Antibody-directed enzyme prodrug therapy (ADEPT)) or a polypeptide which increases the serum half-life of the antibody.

b) Glycosylation Variants

Antibodies of the present disclosure can, in certain embodiments, be altered to increase or decrease the extent to which the antibody is glycosylated. For example, but not by way of limitation, the addition or deletion of glycosylation sites of an antibody may be conveniently accomplished by altering the amino acid sequence such that one or more glycosylation sites is created or removed.

Where the antibodies of the present disclosure comprise an Fc region, the carbohydrate attached thereto, if present, can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In certain embodiments, modifications of the oligosaccharide in an antibody of the present disclosure can be made in order to create antibody variants with certain improved properties.

In certain embodiments, antibody variants are provided having a carbohydrate structure that lacks fucose attached (directly or indirectly) to an Fc region. For example, the amount of fucose in such antibody can be from about 1% to about 80%, from about 1% to about 65%, from about 5% to about 65% or from about 20% to about 40% and values in between.

In certain embodiments, the amount of fucose can be determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 can also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

Defucosylated antibodies can be produced in any cell line that are deficient in protein fucosylation. Non-limiting examples of cell lines include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Non-limiting examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants can have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications can be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the present disclosure provides antibody variants that possess some but not all effector functions. Such limited effector function can make the antibody variants desirable candidates for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (Cell Technology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Intl. Immunol. 18(12):1759-1769 (2006)). In certain embodiments, alterations can be made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. See, e.g., U.S. Pat. No. 6,737,056; WO 2004/056312, and Shields et al., J. Biol. Chem. 9(2): 6591-6604 (2001).

In certain embodiments, antibody variants of the present disclosure comprise an Fc region with one or more amino acid substitutions that improve ADCC, e.g., substitutions at positions 298, 333, and/or 334 of the Fc region (EU numbering of residues).

In certain embodiments, alteration made in the Fc region of an antibody, e.g., a bispecific antibody, disclosed herein, can produce a variant antibody with an increased half-life and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein, which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies can be generated as described, e.g., in U.S. Pat. No. 7,521,541.

e) Antibody Derivatives

In certain embodiments, antibodies of the present disclosure can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). In certain embodiments, the radiation can be of any wavelength, and includes, but is not limited to, wavelengths that do not harm ordinary cells, but which heat the nonproteinaceous moiety to a temperature at which cells proximal to the antibody-nonproteinaceous moiety are killed.

B. Methods of Antibody Production

The antibodies, e.g., capture and/or detection antibodies, disclosed herein can be produced using any available or known technique in the art. For example, but not by way of limitation, antibodies can be produced using recombinant methods and compositions, e.g., as described in U.S. Pat. No. 4,816,567. Detailed procedures to generate antibodies are described in the Examples below.

The presently disclosed subject matter further provides an isolated nucleic acid encoding an antibody disclosed herein. For example, the isolated nucleic acid can encode an amino acid sequence that includes the VL and/or an amino acid sequence comprising the VH of the antibody, e.g., the light and/or heavy chains of the antibody.

In certain embodiments, the nucleic acid can be present in one or more vectors, e.g., expression vectors. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the disclosed subject matter is intended to include such other forms of expression vectors, such as viral vectors (e.g., replication defective retroviruses, adenoviruses and adeno-associated viruses) that serve equivalent functions.

In certain embodiments, the nucleic acid encoding an antibody of the present disclosure and/or the one or more vectors including the nucleic acid can be introduced into a host cell. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, a host cell can include, e.g., has been transformed with: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In certain embodiments, the host cell is eukaryotic, e.g., a Chinese Hamster Ovary (CHO) cell or lymphoid cell (e.g., Y0, NS0, Sp20 cell).

In certain embodiments, the methods of making a disclosed anti-CD20 antibody can include culturing a host cell, in which a nucleic acid encoding the antibody has been introduced, under conditions suitable for expression of the antibody, and optionally recovering the antibody from the host cell and/or host cell culture medium. In certain embodiments, the antibody is recovered from the host cell through chromatography techniques.

For recombinant production of an antibody of the present disclosure, a nucleic acid encoding an antibody, e.g., as described above, can be isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies can be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006). Suitable host cells for the expression of glycosylated antibody can also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

In certain embodiments, plant cell cultures can be utilized as host cells. See, e.g., U.S. Pat. Nos. 5,959,177, 6,040,498, 6,420,548, 7,125,978, and 6,417,429 (describing PLANTIBODIES™ technology for producing antibodies in transgenic plants).

In certain embodiments, vertebrate cells can also be used as hosts. For example, but not by way of limitation, mammalian cell lines that are adapted to grow in suspension can be useful. Non-limiting examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In certain embodiments, techniques for making bispecific and/or multispecific antibodies include, but are not limited to, recombinant co-expression of two immunoglobulin heavy chain-light chain pairs having different specificities (see Milstein and Cuello, Nature 305: 537 (1983)), PCT Patent Application No. WO 93/08829, and Traunecker et al., EMBO J. 10: 3655 (1991)), and “knob-in-hole” engineering (see, e.g., U.S. Pat. No. 5,731,168). Bispecific antibodies can also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Bispecific and multispecific molecules of the present disclosure can also be made using chemical techniques (see, e.g., Kranz (1981) Proc. Natl. Acad. Sci. USA 78:5807), “polydoma” techniques (see, e.g., U.S. Pat. No. 4,474,893) or recombinant DNA techniques. Bispecific and multispecific molecules of the presently disclosed subject matter can also be prepared by conjugating the constituent binding specificities, e.g., a first epitope and a second epitope binding specificities, using methods known in the art and as described herein. For example, but not by way of limitation, each binding specificity of the bispecific and multispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohaxane-1-carboxylate (sulfo-SMCC) (see, e.g., Karpovsky (1984) J. Exp. Med. 160:1686; Liu (1985) Proc. Natl. Acad. Sci. USA 82:8648). Other methods include those described by Paulus (Behring Ins. Mitt. (1985) No. 78, 118-132; Brennan (1985) Science 229:81-83), Glennie (1987) J. Immunol. 139: 2367-2375). When the binding specificities are antibodies (e.g., two humanized antibodies), they can be conjugated via sulfhydryl bonding of the C-terminus hinge regions of the two heavy chains. In certain embodiments, the hinge region can be modified to contain an odd number of sulfhydryl residues, e.g., one, prior to conjugation.

In certain embodiments, both binding specificities of a bispecific antibody can be encoded in the same vector and expressed and assembled in the same host cell. This method is particularly useful where the bispecific and multispecific molecule is a MAb×MAb, MAb×Fab, Fab×F(ab′)₂ or ligand x Fab fusion protein. In certain embodiments, a bispecific antibody of the present disclosure can be a single chain molecule, such as a single chain bispecific antibody, a single chain bispecific molecule comprising one single chain antibody and a binding determinant or a single chain bispecific molecule comprising two binding determinants. Bispecific and multispecific molecules can also be single chain molecules or can comprise at least two single chain molecules. Methods for preparing bi- and multispecific molecules are described, for example, in U.S. Pat. Nos. 5,260,203; 5,455,030; 4,881,175; 5,132,405; 5,091,513; 5,476,786; 5,013,653; 5,258,498; and 5,482,858. Engineered antibodies with three or more functional antigen binding sites (e.g., epitope binding sites) including “Octopus antibodies,” are also included herein (see, e.g., US 2006/0025576A1).

In certain embodiments, an animal system can be used to produce an antibody of the present disclosure. One animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known (see, e.g., Harlow and Lane (1988), Antibodies, A Laboratory Manual, Cold Spring Harbor Laboratory Press, Cold Spring Harbor N.Y.).

### IV. KITS

The presently disclosed subject matter further provides kits containing materials useful for performing the immunoassays disclosed herein. In certain embodiments, the kit includes a container containing an antibody, e.g., an anti-CD20 antibody, disclosed herein. Non-limiting examples of suitable containers include bottles, test tubes, vials and microtiter plates. The containers can be formed from a variety of materials such as glass or plastic. In certain embodiments, the kit further includes a package insert that provides instructions for using the antibody, e.g., anti-CD20 antibody, in the disclosed immunoassay methods.

In certain embodiments, the kit can include one or more containers containing one or more antibodies. For example, but not by way of limitation, the kit can include at least one container that includes a capture antibody and at least one container that includes a detector antibody.

In certain embodiments, a kit for detecting tumor antigen protein in a sample includes a first container containing a capture antibody that binds to an epitope present within the amino acid sequence of the target protein, a second container containing a detector antibody that binds to an epitope present within the amino acid sequence of the target protein and a third container containing a detection agent. In some embodiments, the capture antibody and detector antibody bind to different epitopes present within the amino acid sequence of the target protein.

In certain embodiments, the capture and/or detection antibody is selected from the group consisting of rituximab, ocrelizumab, ofatumumab, obinutuzumab, a CD20 T-Cell dependent bispecific antibody, and combinations thereof.

In certain embodiments, the capture antibody and/or the detector antibody can be provided in a kit of the present disclosure at a concentration of about 0.1 μg/ml to about 5.0 μg/ml. For example, the capture antibody and/or the detector antibody can be provided in an ELISA kit at a concentration of about 0.1 μg/ml to about 5.0 μg/ml. In non-limiting embodiments, the capture antibody and/or the detector antibody can be provided in a Quanterix kit a concentration of about 0.1 μg/ml to about 2.0 μg/ml. In certain embodiments, the detector antibody can be labeled, e.g., with biotin.

In certain embodiments, the detection agent provided in a kit of the present disclosure can be avidin, streptavidin-HRP or streptavidin-β-D-galactopyranose (SBG). In certain embodiments, a kit of the present disclosure can further include tetramethylbenzidine, hydrogen peroxide and/or resorufin β-D-galactopyranoside. In certain embodiments, if the kit includes streptavidin-HRP, then the kit can further include tetramethylbenzidine and hydrogen peroxide. In certain embodiments, if the kit includes SBG, then the kit can further include resorufin β-D-galactopyranoside. In certain embodiments, SBG can be provided in a kit at a concentration from about 100 pM to about 400 pM.

In certain embodiments, the capture antibody can be provided attached to solid support surface, such as, for example but not limited to, a plate or a bead, e.g., a paramagnetic bead. Alternatively or additionally, the kit can further include a solid support surface that can be coupled to the capture antibody. In certain embodiments, the solid support can be paramagnetic beads and can be provided at a concentration from about 0.1×10⁷ beads/ml to about 10.0×10⁷ beads/ml.

Alternatively or additionally, the kit can include other materials desirable from a commercial and user standpoint, including other buffers, diluents and filters. In certain embodiments, the kit can include materials for collecting and/or processing blood samples.

The following examples are merely illustrative of the presently disclosed subject matter and should not be considered as limitations in any way.

### IV. EXEMPLARY EMBODIMENTS

A1. In certain non-limiting embodiments, the present disclosure provides for an assay for detecting a membrane-associated protein in a sample comprising: a capture antibody that binds to an extracellular vesicle comprising the membrane-associated protein in the sample thereby generating a capture antibody-extracellular vesicle complex; and b) a detection antibody that binds to the capture antibody-extracellular vesicle complex to form a detectable bound complex, wherein the signal from the detectable bound complex is calibrated against one or more known values detected from extracellular vesicle comprising the protein.

A2. In certain embodiments of A1, the capture antibody does not compete for binding with the detection antibody.

A3. In certain embodiments of A1 and A2, the capture antibody binds a different epitope than the detection antibody.

A4. In certain embodiments of A1-A3, the membrane-associated protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

A5. In certain embodiments of A1-A4, the capture antibody is selected from the group consisting of: rituximab, ocrelizumab, ofatumumab, obinutuzumab, and combinations thereof.

A6. In certain embodiments of A1-A5, the detection antibody is selected from the group consisting of: rituximab, ocrelizumab, ofatumumab, obinutuzumab, and combinations thereof.

A7. In certain embodiments of A1-A6, the assay further comprises an extracellular vesicle calibrator.

A8. In certain embodiments of A1-A7, the sample is selected from the group consisting of a plasma sample, a serum sample, a tissue culture supernatant sample, and combinations thereof.

B1. In certain non-limiting embodiments, the present disclosure is directed to a method for quantifying the concentration of circulating protein in a sample comprising the steps of: a) determining the level of a target protein in extracellular vesicles in the sample; and b) comparing the level of the target protein in the extracellular vesicles in the sample with a calibration curve generated using extracellular vesicles comprising the target protein.

B2. In certain embodiments of B1, the target protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

B3. In certain embodiments of B1 or B2, the sample is selected from the group consisting of a plasma sample, a serum sample, a tissue culture supernatant sample, and a combination thereof.

B4. In certain embodiments of B1-B3, the concentration of the target protein and the calibration curve are determined using an immunoassay, an ELISA and/or a Western Blot.

B5. In certain embodiments of B1-B4, the method further comprises detecting the presence of an extracellular vesicle marker, wherein the extracellular marker is selected from the group consisting of CD81, CD63, CD9, and combination thereof.

C1. In certain non-limiting embodiments, the present disclosure is directed to a method for quantifying the concentration of circulating protein in a sample comprising the steps of: a) generating a calibration curve using extracellular vesicles comprising the protein, and b) comparing the level of the protein in extracellular vesicles in the sample with the calibration curve to determine the quantity of the protein in the extracellular vesicles in the sample.

C2. In certain embodiments of C1, the protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

C3. In certain embodiments of C1 or C2, the sample is selected from the group consisting of: a plasma sample, a serum sample, a tissue culture supernatant sample, and combination thereof.

C4. In certain embodiments of C1-C3, the concentration of the circulating protein and the calibration curve are determined using an immunoassay, an ELISA, and/or a Western Blot.

C5. In certain embodiments of C1-C4, the method further comprises detecting the presence of an extracellular vesicle marker, wherein the extracellular marker is selected from the group consisting of CD81, CD63, CD9, and combination thereof.

D1. In certain non-limiting embodiments, the present disclosure is directed to a method for determining whether a patient with a B-cell lymphoma is likely to exhibit a response to an anti-CD20 therapy, comprising the steps of: a) obtaining a sample from the patient; b) determining the quantity of circulating CD20 in extracellular vesicles in the sample; c) comparing the level of CD20 in the extracellular vesicles in the sample with a calibration curve generated using extracellular vesicles comprising CD20; and d) determining whether the patient is likely to exhibit a response to the CD20 therapy, based on the quantity of circulating CD20 in extracellular vesicles determined in the sample.

D2. In certain embodiments of D1, the anti-CD20 therapy comprises administration of an anti-CD20 antibody.

D3. In certain embodiments of D1 or D2, the anti-CD20 antibody is selected from the group consisting of: rituximab, ocrelizumab, ofatumumab, obinutuzumab, a CD20 T-Cell dependent bispecific antibody, and combinations thereof.

D4. In certain embodiments of D1-D3, the sample is selected from the group consisting of a plasma sample, a serum sample, a tissue culture supernatant sample, and combinations thereof.

D5. In certain embodiments of D1-D4, the concentration of the circulating protein and the calibration curve are determined using an immunoassay, an ELISA, and/or a Western Blot.

D6. In certain embodiments of D1-D5, the method further comprises detecting a presence of an extracellular marker, wherein the extracellular marker is selected from the group consisting of CD81, CD63, CD9, and combination thereof.

E1. In certain non-limiting embodiments, the present disclosure is directed to a method for determining the affinity of an anti-CD20 antibody comprising, subjecting the anti-CD20 antibody to a surface plasmon resonance (SPR) analysis, wherein the SPR analysis comprises use of extracellular vesicles expressing CD20 as a ligand and the anti-CD20 antibody as an analyte.

E2. In certain embodiments of E1, the anti-CD20 antibody is selected from the group consisting of: rituximab, ocrelizumab, ofatumumab, obinutuzumab, a CD20 T-Cell dependent bispecific antibody, and combinations thereof.

E3. In certain embodiments of E1-E2, the method further comprises detecting the presence an extracellular marker, wherein the extracellular marker is selected from the group consisting of CD81, CD63, CD9, and combination thereof.

F1. In certain non-limiting embodiments, the present disclosure is directed to a method for determining the activation of T cells obtained from a patient comprising: a) incubating extracellular vesicles expressing CD20 with T cells and a CD20 T-cell dependent bispecific antibody; and b) determining the activation of T cells.

F2. In certain embodiments of F1, the method further comprises detecting the presence of an extracellular marker, wherein the extracellular marker is selected from the group consisting of CD81, CD63, CD9, and combination thereof.

G1. In certain non-limiting embodiments, the present disclosure is directed to a method of treating a tumor in a subject in need comprising: a) obtaining a sample from the subject; b) generating a calibration curve using extracellular vesicles comprising a tumor antigen; c) comparing a level of the tumor antigen in extracellular vesicles in the sample with the calibration curve to determine the quantity of the target tumor antigen in the extracellular vesicles in the sample; d) determining whether the subject is likely to exhibit a response to an antibody therapy, based on the level of the tumor antigen in extracellular vesicles in the sample; and e) administering a therapeutic in response to the determination in d).

G2. In certain embodiments of G1, the method further comprises detecting the presence of an extracellular marker, wherein the extracellular marker is selected from the group consisting of CD81, CD63, CD9, and combination thereof.

G3. In certain embodiments of G1-G2, the antibody is selected from the group consisting of: rituximab, ocrelizumab, ofatumumab, obinutuzumab, and combinations thereof.

G4. In certain embodiments of G1-G3, the target tumor antigen is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combination thereof.

G5. In certain embodiments of G1-G4, the sample is selected from the group consisting of a plasma sample, a serum sample, a tissue culture supernatant sample, and combinations thereof.

G6. In certain embodiments of G1-G5, the concentration of the circulating tumor antigen and the calibration curve is determined using an immunoassay, an ELISA, and/or a Western Blot.

### EXAMPLES

### Example 1. Preparation of a Tumor Antigen Assay Calibration Curve

A. Culture of Cells Expressing Tumor Antigen

Seed Strain Maintenance: Seed train was passaged every 3 to 4 days. For a 3-day passage, cells were seeded at 0.8×10⁶ cells/mL in a 32% fill (ex. 80 mL fill in 250 mL nonbaffled shake flask) or a 50% fill (ex. 1 L fill in 2 L nonbaffled shake flask) Expi293 expression media in a nonbaffled shake flask; agitate at 125 rpm (32% fill) or 160 rpm (50% fill), 25 mm orbital diameter and incubate at 8% CO2, 80% humidity, 37° C.; cells should grow to >4×10⁶ cells/mL, >95% viability. For a 4-day passage, cells were seeded at 0.4×10⁶ cells/mL in a 32% fill (ex. 80 mL fill in 250 mL nonbaffled shake flask) or a 50% fill (ex. 1 L fill in 2 L nonbaffled shake flask) Expi293 expression media in a nonbaffled shake flask; agitate at 125 rpm (32% fill) or 160 rpm (50% fill), 25 mm orbital diameter and incubate at 8% CO2, 80% humidity, 37° C.; cells should grow to >4×10⁶ cells/mL, >95% viability.

Seed production cultures: Expi293 seed train was cultured in Hyclone HyCell TransFX-H media, 10 mg/mL gentamicin (A466), 10% pluronic F-68, 20 mM L-Glutamine (A0821). A container and 125 mL shake flasks/50 mL tubespins were used for dilution.

Count seed train culture for viable cell density and viability (at >4×10⁶ cells/mL, >95% viability): volume of culture needed for transfections was calculated (V_{F}=30 mL×#of transfections).

Prepare production media: Hyclone media was supplemented with, 0.5 g/L pluronic F-68 (add 5 mL of 10% pluronic F-68 to 1 L Hyclone media); 4 mM L-Glutamine (add 20 mL 20 mM L-Glutamine (A0821) to 1 L Hyclone media); and 0.21 g/L gentamicin (add 21 mL of 10 mg/mL gentamicin (A466) to 1 L Hyclone media)(optional).

Expi293 seed train was diluted to 2.0×10⁶ cells/mL in appropriate amount of production media; this is the culture that will be transfected. The following formulas were used to calculate seed train culture needed for dilution:

$${V}_{C} = \frac{{X}_{F} {V}_{F}}{{X}_{C}}$$

Where:

V_{C}=seed train culture volume needed (mL)

X_{F}=final desired viable cell density for transfections (2.0×10⁶ cells/mL)

V_{F}=final culture volume required for all transfections (mL)

X_{C}=seed train culture viable cell density (cells/mL)

The Expi293 production culture dilution was aliquoted 25.5 mL per flask/tubespin. Place flasks/tubespins at 37° C., 8% CO2, 125 rpm (flasks at 25 mm orbital diameter) or 225 rpm (tubespins at 50 mm orbital diameter) and allow to equilibrate (at least 15 minutes).

B. Extracellular Vesicle Tumor Antigen Calibrator Purification Protocol

pB_EF1_hCD20 construct transfected Expi293 7-day culture was collected and spun down at 500 g for 10 min. Supernatant was decanted into another 50 ml conical and spun at 2000 g for 10 min. The Supernatant was decanted into a 0.22 um vacuum filter and filtered. The filtered media was concentrated with 70 ml centrifugation concentrators (Centricon Plus-70, UFC710008): loaded 60 ml supernatant, 3750 rpm at 4° C. for 10 min, gentle mixed supernatant; 3750 rpm at 4° C. for 10 min; decanted the filtrate, added more supernatant, spun more times until the volume is less than 12 ml. The concentrate was recovered at 750 g (max 1000 g) at 4° C. for 2 min. The concentrate was spun no more than 10 min to avoid precipitation and aggregation. The concentrated media was spun in ultracentrifuge at 30k rpm at 4° C. for 75 min. Tubes were properly balanced within 0.01 g with PBS including the ones without samples (Use H₂O to balance). Accel and Decel both used Max. The supernatant was decanted. A pellet should be visible at the bottom of the tube. The pellet was re-suspended in 500 uL PBS and then re-filled ultracentrifuge tubes with 12 mL 1×PBS. The mixture was balanced with PBS again and re-centrifuged at 30k rpm (100,000 g) at 4° C. for 75 min. The supernatant was poured off and pellets were gently re-suspended in 0.5 to 1 ml PBS.

C. EV Tumor Antigen Calibrator Value Assignment by Western Blot

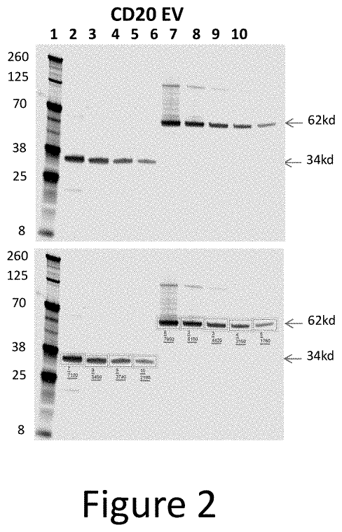

FIG. 2 depicts an exemplary characterization of an EV tumor antigen calibrator prepared as described herein, where the values are assigned by western blot. Column 1 represents a marker, column 2 represents EV 2 ug, column 3 represents EV 1 ug, column 4 represents EV 0.5 ug, column 5 represents EV 0.25 ug, column 6 represents rhCD20 250 ng, column 7 represents rCD20 100 ng, column 8 represents rCD20 40 ng, column 9 represents rCD20 16 ng, and column 10 represents rCD20 6.4 ng.

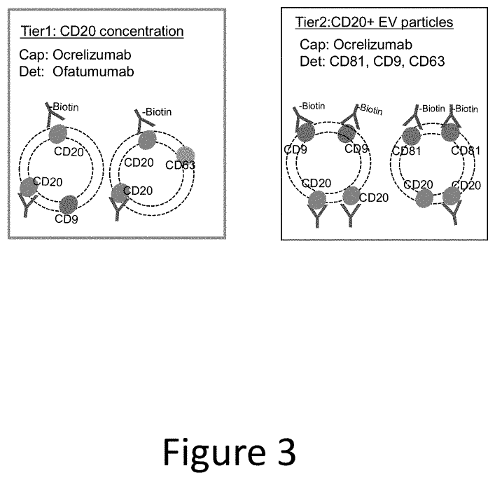

FIG. 3 demonstrates the presence of CD20 in plasma samples from normal and NHL (e.g., DLBCL and FL) donors using anti-CD20 Ab as capture and anti-CD20 as detection or anti-tetraspanin antibodies as detection. Tetraspanin antibodies also can be used to detect the presence of CD20 in extracellular vesicles. For example, capture using CD20 and detection using CD81, CD9, CD63 demonstrated that these markers were co-localized and CD20 was present in membrane (or extracellular vesicles). Since not all vesicles have all or same markers, therefore, cocktail was needed for detection.

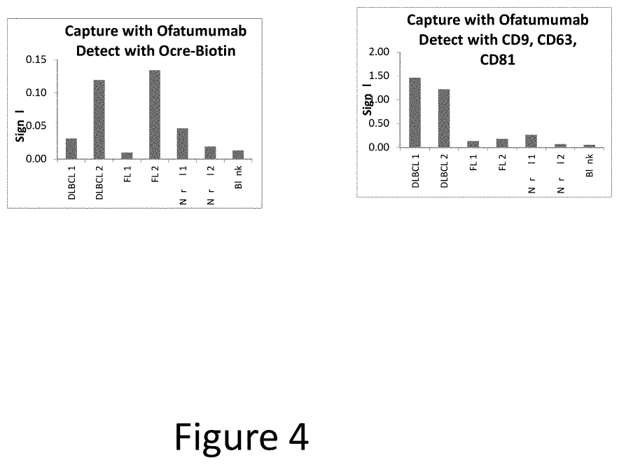

FIG. 4 shows an exemplary ELISA format when anti-CD20 antibodies are used to detect CD 20 in plasma from normal and NHL (e.g., DLBCL and FL). ELISA data in FIG. 4 shows evidence of co-localization of these markers in neat plasma without ultracentrifugation.

D. EV Tumor Antigen Standard Curve by Quanterix

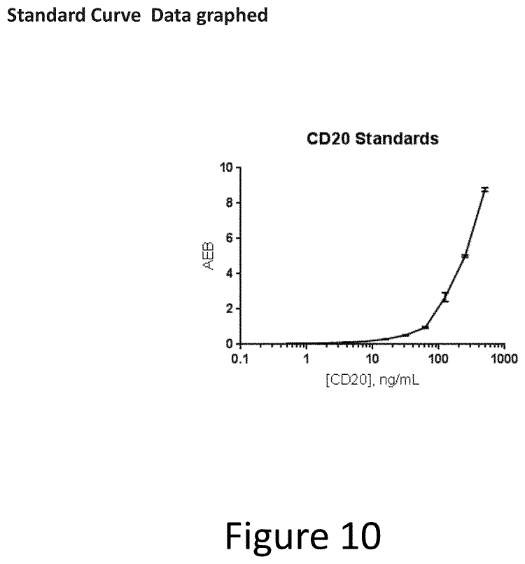

The following materials were used for Quanterix assay: a) Standard curve and sample diluent PBS, 1.5% BSA, 0.05% Polysorbate 20, 0.05% Proclin 300, pH 7.4, b) Anti-DIG antibody coupled beads, c) DIG coupled ofatumumab as capture antibody for CD20 antigen, d) biotin coupled ofatumumab as detection antibody, e) Streptavidin coupled beta galactosidase (SBG) as an enzyme reagent, and f) RGP, a substrate for SBG that used as reporter for signal (See also FIG. 10)

CD20 expressed in extra cellular vesicles was diluted in standard curve diluent at a starting concentration of 500 ng/mL. Ten 2-fold serial dilutions are made to a final concentration of 0.5 ng/mL. The eleven levels plus a non-specific blank were pipetted into the Quanterix polyproyilene low binding plate. The detector and capture antibodies diluted into PBS, 1.5% BSA, 0.05% Polysorbate 20, 0.05% Proclin 300, pH 7.4. to 0.5 ug/mL were prepared and loaded onto the instrument before the 96-well plate. The enzyme reagent SA Beta Galactosidase was diluted in its own buffer to a conc of 150 pM. The raw data was downloaded from the instrument in a excel cvs file and regressed using SoftMax Pro software with a 5 pl fit. Table 2 provides an exemplary EV CD20 standard curve by Quanterix.

TABLE 2
EV CD20 Standard Curve by Quanterix

| Nominal | | | Assayed | CV (Assayed | Recovery |
|---|---|---|---|---|---|
| Concen- | | AEB | Concen- | Concen- | from |
| tration | | CV | tration | tration) | Nominal |
| (ng/ml) | AEB | (%) | (ng/ml) | (%) | (%) |
| Non-specific | 0.048 | 5.635 | | | |
| Signal | | | | | |
| 0.488 | 0.058 | 1.0 | 0.24 | 22.6 | 49 |
| 0.977 | 0.066 | 5.0 | 0.91 | 29.3 | 92.9 |
| 1.953 | 0.083 | 4.0 | 2.16 | 11.5 | 111 |
| 3.906 | 0.115 | 7.7 | 4.51 | 13.7 | 116 |
| 7.813 | 0.167 | 2.8 | 8.00 | 3.9 | 102 |
| 15.625 | 0.292 | 3.5 | 15.9 | 3.9 | 102 |
| 31.25 | 0.517 | 4.7 | 29.4 | 4.9 | 94.1 |
| 62.5 | 0.964 | 4.2 | 54.8 | 4.1 | 87.8 |
| 125 | 2.679 | 9.4 | 147 | 9.1 | 118 |
| 250 | 5.007 | 1.2 | 271 | 1.2 | 109 |
| 500 | 8.773 | 1.2 | 478 | 1.3 | 95.6 |

### Example 2. Detection of Tumor Antigen with & without Calibration Curve

Plasma collection. 6 mL of whole blood was collected and transferred into the plasma lavender top Vacutainer tube (plasma collection tube, BD #367863). The collection tube was completely filed until blood flow stops. After the whole blood collection, the plasma lavender top Vacutainer tube 5 times was gently and completely inverted to mix uniformly. The red blood cells were not ruptured by inverting tubes vigorously. Cell lysis can lead to specimen degradation. Specimens were placed on wet ice immediately after blood collection. The process begum within 30 minutes of the blood draw.

Samples were immediately frozen after the processing. The Vacutainer tube was centrifuged at 1600×g for 15 minutes at 4° C. Without disturbing the white layer of cells, the plasma was slowly and carefully collected from the top layer of the tube (˜3 mL) using a transfer pipette and transferred into the pre-labeled two 4.5 mL NUNC tubes. The remaining cell pellet was appropriately discarded. Not all the possible plasma was removed. The plasma stayed about 5 mm away from the buffy coat to avoid contamination of plasma with cellular material (mononuclear cells). The plasma was mixed by inversion 5-6 times and aliquoted in pre-labeled 2.0 mL Sarstedt tubes. The samples were transferred in upright position to −70/−80° C. freezer (preferred) or −20° C. freezer (alternate) for storage.

The samples were stored in at −70/−80° C. (preferred) or −20° C. (alternate) until analysis.

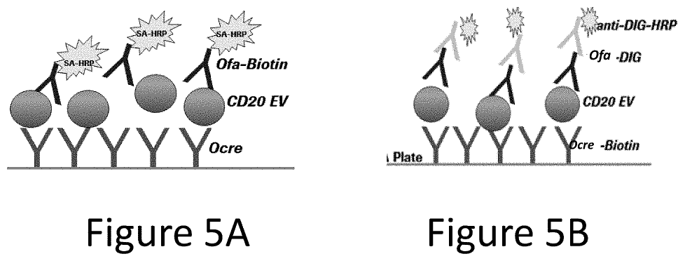

Sequential Assay. A three-day sequential assay (FIG. 5A) can be used in instances where improved sensitivity is desirable. Day 1: Plates were coated overnight at 4° C. with capture antibody. Day 2: Samples were added and incubated overnight at 4° C. Day 3: Detection antibody (conjugated to biotin) and SA-HRP were added. The following antibody and signal conditions were used: Ocre 1 ug/ml (capture antibody), Ofa 0.5 ug/ml (detection antibody) and HRP 100 ng/mL (signal). Table 3 provides an exemplary data created by the sequential assay.

TABLE 3
Parallelism: Three Day Sequential Assay

| | | | Dilution | | |
|---|---|---|---|---|---|
| | | | corrected | % recovery | % recovery |
| Sample | Dilution | Absorbance | (ng/mL) | from neat | from 2 fold |
| D8 | neat | 0.63 | 153.5 | Ref | — |
| | 2 | 0.403 | 190 | 124 | Ref |
| | 4 | 0.25 | 219.2 | 115 | 115 |
| | 8 | 0.161 | 240.7 | 110 | 110 |
| | 16 | 0.335 | 1237.4 | 514 | 514 |
| F9 | neat | 2.432 | 816.466 | Ref | — |
| | 2 | 1.745 | 991.9 | 121 | Ref |
| | 4 | 0.902 | 904.2 | 91 | 91 |
| | 8 | 0.425 | 804.7 | 89 | 89 |
| | 16 | 0.203 | 672.7 | 84 | 84 |
| F10 | neat | 0.911 | 228.397 | Ref | — |
| | 2 | 0.524 | 252.1 | 110 | Ref |
| | 4 | 0.281 | 252.4 | 100 | 100 |
| | 8 | 0.139 | 185.9 | 74 | 74 |
| | 16 | 0.099 | 164.1 | 88 | 88 |
| F3 | neat | 1.031 | 262.143 | Ref | — |
| | 2 | 0.626 | 305 | 116 | Ref |
| | 4 | 0.404 | 380.9 | 125 | 125 |
| | 8 | 0.192 | 311.6 | 82 | 82 |
| | 16 | 0.116 | 256 | 82 | 88 |
| F6 | neat | 0.767 | 189.456 | Ref | — |
| | 2 | 0.556 | 268.7 | 142 | Ref |
| | 4 | 0.303 | 276.3 | 103 | 103 |
| | 8 | 0.221 | 375.4 | 136 | 136 |
| | 16 | 0.158 | 463.9 | 124 | 124 |
| F1 | neat | 0.777 | 192.092 | Ref | — |
| | 2 | 0.519 | 249.6 | 130 | Ref |
| | 4 | 0.311 | 284.1 | 114 | 114 |
| | 8 | 0.392 | 737.9 | 260 | 260 |
| | 16 | 0.15 | 428.6 | 58 | 58 |

Bridging Assay. A two-day bridging assay (FIG. 5B) can be used in instances where reduced time to results is desirable. Day 1: 100 uL sample was incubated with 100 uL master mix (Ab-DIG+Ab-Biotin) overnight at 4° C. Day 2: The 100 uL of sample with master mix was removed and transferred to a SA plate. Signals were detected with anti-DIG-HRP. The following antibody and signal conditions were used: master mix 1 ug/mL (of a-DIG+anti-DIG-biotin) and HRP 50 ng/mL (signal). Table 4 provides an exemplary data created by the bridging assay.

TABLE 4
Parallelism: Two Day Bridging Assay

| | | | | | % Recovery |
|---|---|---|---|---|---|
| | Fold | | Obs Conc | % Recovery | (from ½ |
| Sample | Dilution | Signal | [ng/ml] | (from Neat) | MRD) |
| F1 | 1 | 0.228 | 81.40 | Ref | — |
| | 2 | 0.118 | 39.00 | 96% | Ref |
| | 4 | 0.080 | 23.61 | 116% | 121% |
| | 8 | 0.55 | 13.77 | 135% | 141% |
| F2 | 1 | 0.322 | 117.92 | — | — |
| | 2 | 0.168 | 58.15 | 99% | — |
| | 4 | 0.109 | 35.43 | 120% | 122% |
| | 8 | 0.084 | 25.37 | 172% | 175% |
| F3 | 1 | 2.649 | 1647.70 | Ref | — |
| | 2 | 2.054 | 1030.81 | 125% | Ref |
| | 4 | 1.189 | 494.65 | 120% | 96% |
| | 8 | 0.740 | 287.90 | 140% | 112% |
| D1 | 1 | 0.137 | 43.95 | — | — |
| | 2 | 0.062 | 13.99 | 64% | — |
| | 4 | 0.046 | 8.22 | 75% | 117% |
| | 8 | 0.033 | 3.57 | 65% | 102% |
| D2 | 1 | 0.242 | 89.46 | Ref | — |
| | 2 | 0.147 | 48.18 | 108% | Ref |
| | 4 | 0.093 | 25.88 | 116% | 107% |
| | 8 | 0.070 | 17.01 | 152% | 141% |
| D3 | 1 | 0.053 | 10.67 | — | — |
| | 2 | 0.032 | 3.44 | 64% | — |
| | 4 | 0.025 | 1.26 | 47% | 73% |
| | 8 | 0.028 | 1.96 | 147% | 228% |
| D4 | 1 | 0.101 | 29.23 | Ref | — |
| | 2 | 0.066 | 15.65 | 107% | Ref |
| | 4 | 0.040 | 5.93 | 81% | 76% |
| | 8 | 0.032 | 3.24 | 89% | 83% |

Signal Detection without Calibration Curve: samples and reagents were diluted in standard curve diluent without EV calibrator. Samples were serially diluted 2-fold. Diluted samples were pipetted into the Quanterix polypropylene low binding plate. Bead coupled to anti-DIG antibody, Ofatumumab-DIG and Ofatumumab-biotin are diluted into standard curve diluent to a concentration of 0.5 ug/mL. Beads are diluted to a nominal bead conc of 1.4×10⁹ beads/mL. The enzyme (streptavidin ß-galactosidase, SBG), was diluted into SBG diluent to 150 pM. The beads, detectors, enzyme, substrate and 96-well plate containing standards/samples were loaded onto the instrument. The raw data was downloaded from the instrument in a excel cvs file. The raw data was processed using Excel spreadsheet.

As shown in Table 5, no calibration curve was generated with recombinant human, where ocrelizumab was used as capture antibody and ofatumumab was used as detection antibody. Commercially available recombinant human did not elicit signal with Ocre/Ofa combination possibly due to lack of formation of loop by transmembrane helices. Linear peptide or recombinant protein not bound to membrane does not elicit signal in ELISA suggesting that ocrelizumab or ofatumumab don't bind to CD20 in that conformation.

TABLE 5
Detection of Signal Without Calibration Curve

| Ab pair: | Rh CD20 Exp | Signal Full | Signal | Signal |
|---|---|---|---|---|
| Ocre/Ofa | Conc. (ng/ml) | Length | Truncated 1 | Truncated 2 |
| 1 | 200 | 0.01 | 0.01 | 0.01 |
| 2 | 66.67 | 0.01 | 0.01 | 0.01 |
| 3 | 22.22 | 0.01 | 0.01 | 0.01 |
| 4 | 7.41 | 0.01 | 0.01 | 0.01 |
| 5 | 2.47 | 0.01 | 0.02 | 0.01 |
| 6 | 0.82 | 0.01 | 0.01 | 0.01 |
| 7 | 0.27 | 0.01 | 0.01 | 0.01 |
| 8 | 0 | 0.01 | 0.01 | 0.01 |

Signal Detection with Calibration Curve: CD20 EV was diluted in standard curve diluent at a starting concentration of 500 ng/mL. Ten 2-fold serial dilutions were made to a final concentration of 0.5 ng/mL. The eleven levels plus a non-specific blank were pipetted into the Quanterix polypropilene low binding plate.

Ofatumumab-DIG and Ofatumumab-biotin were diluted into BA003+1.5% BSA to a concentration of 0.5 ug/mL. Anti-DIG Ab-beads are diluted into BA003+1.5% BSA to a nominal bead conc of 1.4×10⁹ beads/mL. The enzyme (streptavidin ß-galactosidase, SBG) was diluted into SBG diluent to 150 pM. The beads, detectors, enzyme, substrate and 96-well plate containing standards/samples were loaded onto the instrument. Raw data was exported and analyzed using Softmax Pro.

Table 6 provides does-dependent signal (average enzyme per bead, See also FIG. 10), standard deviation, CD observed concentration, coefficient of variation of concentration, and recovery. The following formulas were used:

Signal: Average enzymes per Bead (AEB),

$$Coefficient of Variation ( \% C . V . ) = ( \frac{standarddeviation}{theoretical value} ) \times 100 , \\ Standard Deviation ( S . D . ) = \sqrt{\frac{{Σ ( x - \overline{x} )}^{2}}{n - 1}} , and$$

$$Difference from Theoretical ( Recovery \% ) = [ ( \frac{mean calulatedconcentration}{theoretical concentration} ) - 1 ] \times 100$$

TABLE 6
Detection of CD20 EV Signal With anti-CD20 antibodies using Calibration Curve

| | CD20 | | | | | |
|---|---|---|---|---|---|---|
| | Expected | | | CD Observed | | |
| Std | Concentration | | STD | Concentration | Concentration | % |
| Curve | [ng/ml] | Signal | DEV | [ng/ml] | % CV | Recovery |
| 1 | 500.00 | 2.92 | 0.002 | 498.92 | 0.12 | 99.78 |
| 2 | 250.00 | 1.90 | 0.007 | 252.92 | 0.51 | 101.17 |
| 3 | 125.00 | 1.05 | 0.020 | 121.56 | 2.17 | 97.25 |
| 4 | 62.50 | 0.58 | 0.002 | 62.71 | 0.44 | 100.34 |
| 5 | 31.25 | 0.32 | 0.003 | 32.82 | 1.19 | 105.01 |
| 6 | 15.63 | 0.17 | 0.007 | 16.55 | 4.46 | 105.91 |
| 7 | 7.81 | 0.10 | 0.003 | 8.09 | 3.90 | 103.60 |
| 8 | 3.91 | 0.06 | 0.002 | 4.39 | 4.00 | 112.42 |
| 9 | 1.95 | 0.04 | 0.001 | 1.34 | 6.80 | 68.54 |
| 10 | 0.98 | 0.03 | 0.002 | 0.37 | 56.40 | 37.68 |
| 12 | 0.00 | 0.02 | 0.000 | | | |

### Example 3. Bead-Based Immunoassay Format

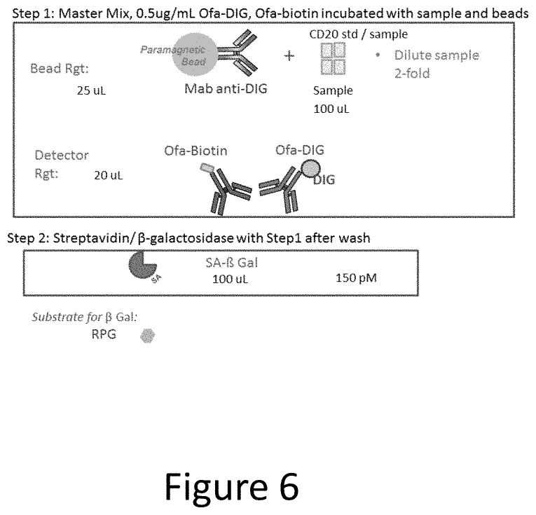

Alternative formats useful for detecting proteins, e.g., tumor antigens, include bead-based immunoassays, e.g., the Quanterix platform. In such an assay anti-DIG Ab followed by Ofatumumab-DIG coating can be used for capturing cCD20 and Ofatumumab-biotin followed by Streptavidin beta galactosidase can be used for detection (FIG. 6).

In the exemplary bead-based immunoassay format, Ofatumumab-DIG and Ofatumumab-biotin were diluted into BA003+1.5% BSA to a concentration of 0.5 ug/mL. Beads labeled with Antibody to Digoxin (anti-DIG-beads) were diluted into BA003+1.5% BSA to a nominal bead conc of 1.4×10⁹ beads/mL. The enzyme (streptavidin ß-galactosidase, SBG), was diluted into SBG diluent to 150 pM. The beads, detectors, enzyme, substrate and 96-well plate containing standards/samples are loaded onto the instrument.

As shown in FIG. 6, in the first step, the sample, anti-DIG beads, Ofatumumab-Biotin and Ofatumumab DIG detectors were pipetted into a cuvette to form a sandwich, for an incubation of approximate 67 cadences (50 minutes). Then, in the second step, the sandwich was labeled with SBG and incubated for 7 cadences (5 minutes). Between steps, a magnet pelleted the beads and followed by a wash step. The beads were re-suspended in resorufin ß-D-galactopyranoside (RGP) substrate, and transferred to a Simoa Disc for imaging. Table 7 provides does-dependent average enzyme per bead (AEB), coefficient of variability (CV), calculated concentration, CV of concentration, recovery, and signal-to-background ratio.

TABLE 7
Quanterix Detection of cCD20

| Expected | | | | | | |
|---|---|---|---|---|---|---|
| Concentration | | | Calculated | CV- | | |
| [CD20] | AEB | CV | Concentration | Concentration | Recovery | |
| (ng/ml) | (signal) | (%) | (ng/ml) | (%) | (%) | S/B |
| 500 | 6.791 | 1.1 | 477.5 | 1.1 | 96 | 194 |
| 250 | 3.877 | 4.9 | 273 | 4.8 | 109 | 111 |
| 125 | 2.135 | 4 | 150.5 | 3.9 | 121 | 61 |
| 62.5 | 0.772 | 1 | 54 | 1 | 86 | 22.1 |
| 31.25 | 0.442 | 6.1 | 30.3 | 6.4 | 97 | 12.6 |
| 15.625 | 0.236 | 0.4 | 15.4 | 0.5 | 99 | 6.74 |
| 7.813 | 0.135 | 2.4 | 7.975 | 2.9 | 102 | 3.86 |
| 3.906 | 0.08 | 2.4 | 3.93 | 3.6 | 101 | 2.29 |
| 1.953 | 0.057 | 1.2 | 2.19 | 2.4 | 112 | 1.63 |
| 0.977 | 0.045 | 9.1 | 1.295 | 24.2 | 133 | 1.29 |
| 0.488 | 0.032 | 4.2 | 0.2555 | 41.4 | 52 | 0.91 |
| Blank | 0.035 | | | | | |

### Example 4. Impact of Detergent on Detectability

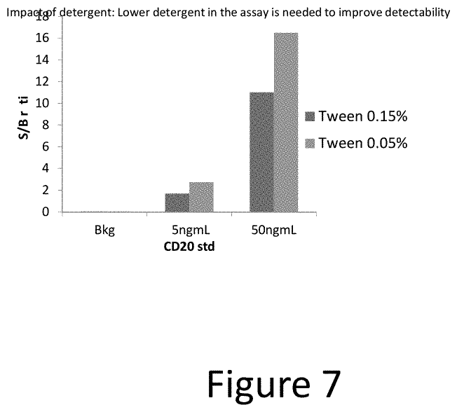

One set of CD20 controls (5 and 50 ng/mL) was prepared in PBS, 1.5% BSA, 0.15% Polysorbate 20, 0.05% Proclin 300, pH 7.4. A second set was prepared in PBS, 1.5% BSA, 0.0 5% Polysorbate 20, 0.05% Proclin 300, pH 7.4. The controls were assayed on Quanterix instrument. As shown in FIG. 7, lower detergent in the assay showed an improved signal-to-background (S/B) ratio.

### Example 5. Drug Tolerance Assay

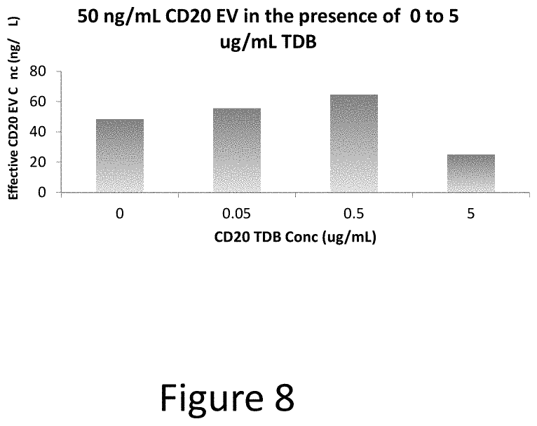

Drug tolerance controls were prepared in a buffer matrix to mitigate endogenous influences (FIG. 8). CD20 TDB and CD20 were each diluted into PBS, 1.5% BSA, 0.05% Polysorbate 20, 0.05% Proclin 300, pH 7.4 at twice the nominal concentration and then combined one to one. Final concentrations were 0, 0.05, 0.5, and 5 ug/mL TDB and 50 ng/mL CD20. Controls were assayed and quantitated against standard curve. The drug tolerance test showed about 50% interference at 50 ng/mL CD20 EV in the presence of 5 ug/mL TDB (e.g., anti-CD20-CD3).

### Example 6. Characterization of Anti-CD20 TDB to CD20 Expressed on Extracellular Vesicles Using Biacore™

Binding interactions between CD20 expressed on extracellular vehicles (EVs) and anti-CD20 TDB were evaluated by surface plasmon resonance (SPR) technology on a Biacore™ T200 instrument (GE Healthcare; Piscataway, N.J.). The dissociation equilibrium constant (K_{D}), dissociation rate constant (kd), and association rate constant (ka) values were calculated with Biacore™ T200 Evaluation Software (Version 3.0; GE Healthcare) using a heterogeneous analyte binding model.

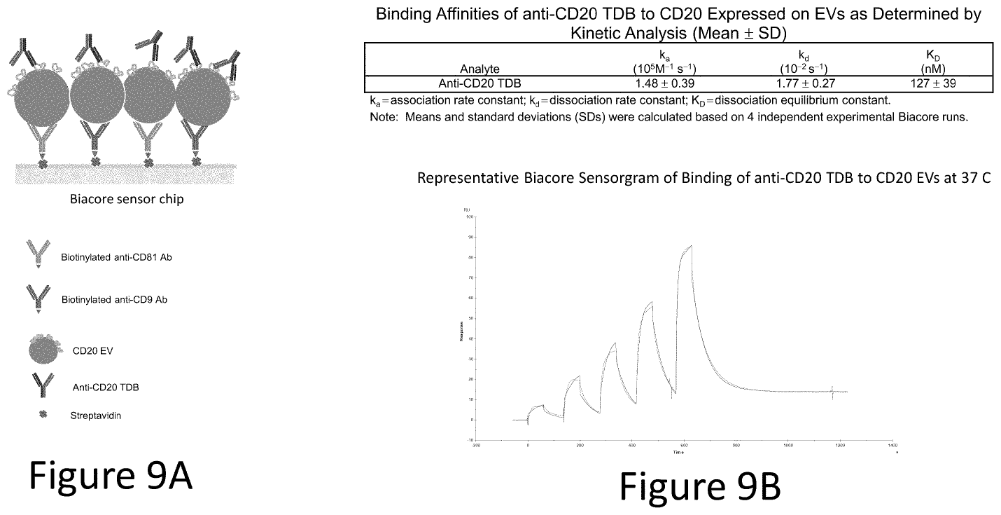

CD20 EVs were captured onto different flow cells (FCs) on a SA sensor chip using an indirect capture method (FIG. 9A). Biotinylated anti-CD81 and anti-CD9 antibodies (mixed with equal concentration of 30 ug/mL) were first captured via Biotin-streptavidin interactions onto all four FCs, resulting in capture level of approximately 2500 response units (RUs). CD20 EVs were then injected over FC2 or FC4 at a concentration of 0.25 μg/mL for 40-120 seconds (s). The resulting capture levels for the EVs ranged from 600-1800 RU. Various concentrations of anti-CD20 TDB were diluted in running buffer (0.01 M HEPES, 0.15 M NaCl, and 3 mM EDTA, pH 7.4) and then injected into the four FCs at a flow rate of 100 μL/min for 1 or 2 minutes (min); the dissociation of anti-CD20 TDB from the antibodies was allowed to proceed for 10 min for kinetic affinity measurements. The experiments were performed at 37° C. and the results are summarized in FIG. 9B. A representative Biacore Sensorgram of Binding of anti-CD20 TBD to CD20 EVs at 37 C is also presented in FIG. 9B.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. An assay for detecting a membrane-associated protein in a sample comprising:
a) a capture antibody that binds to an extracellular vesicle comprising the membrane-associated protein in the sample thereby generating a capture antibody-extracellular vesicle complex, and
b) a detection antibody that binds to the capture antibody-extracellular vesicle complex to form a detectable bound complex,
wherein the signal from the detectable bound complex is calibrated against one or more known values detected from extracellular vesicle comprising the protein.

2. The assay of claim 1, wherein the capture antibody does not compete for binding with the detection antibody.

3. The assay of claim 1, wherein the capture antibody binds a different epitope than the detection antibody.

4. The assay of claim 1, wherein the membrane-associated protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combinations thereof.

5. The assay of claim 1, wherein the capture antibody is selected from the group consisting of rituximab, ocrelizumab, ofatumumab, obinutuzumab, and combinations thereof.

6. The assay of claim 1, wherein the detection antibody is selected from the group consisting of rituximab, ocrelizumab, ofatumumab, obinutuzumab, and combinations thereof.

7. The assay of claim 1, further comprising an extracellular vesicle calibrator.

8. The assay of claim 1, wherein the sample is selected from the group consisting of a plasma sample, a serum sample, a tissue culture supernatant sample, and combinations thereof.

9. A method for quantifying a concentration of circulating protein in a sample comprising the steps of:
a) determining the level of a target protein in extracellular vesicles in the sample, and
b) comparing the level of the target protein in the extracellular vesicles in the sample with a calibration curve generated using extracellular vesicles comprising the target protein.

10. The method of claim 9, wherein the target protein is selected from the group consisting of: a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combinations thereof.

11. The method of claim 9, wherein the sample is selected from the group consisting of a plasma sample, a serum sample, a tissue culture supernatant sample, and a combination thereof.

12. The method of claim 9, wherein the concentration of the target protein and the calibration curve are determined using an immunoassay, an ELISA and/or a Western Blot.

13. The method of claim 9, further comprising detecting a presence of an extracellular vesicle marker, wherein the extracellular marker is selected from the group consisting of CD81, CD63, CD9, and combinations thereof.

14. A method for determining whether a patient with a B-cell lymphoma is likely to exhibit a response to an anti-CD20 therapy, comprising the steps of:
a) obtaining a sample from the patient,
b) determining the quantity of circulating CD20 in extracellular vesicles in the sample,
c) comparing the level of CD20 in the extracellular vesicles in the sample with a calibration curve generated using extracellular vesicles comprising CD20, and
d) determining whether the patient is likely to exhibit a response to the CD20 therapy, based on the quantity of circulating CD20 in extracellular vesicles determined in the sample.

15. The method of claim 14, wherein the anti-CD20 therapy comprises administration of an anti-CD20 antibody.

16. The method of claim 15, wherein the anti-CD20 antibody is selected from the group consisting of: rituximab, ocrelizumab, ofatumumab, obinutuzumab, a CD20 T-Cell dependent bispecific antibody, and combinations thereof.

17. The method of claim 14, wherein the sample is selected from the group consisting of a plasma sample, a serum sample, a tissue culture supernatant sample, and combinations thereof.

18. The method of claim 14, wherein a concentration of the circulating protein and the calibration curve are determined using an immunoassay, an ELISA, and/or a Western Blot.

19. The method of claim 14, further comprising detecting a presence of an extracellular marker, wherein the extracellular marker is selected from the group consisting of CD81, CD63, CD9, and combinations thereof.

20. A method for determining the affinity of an anti-CD20 antibody comprising, subjecting the anti-CD20 antibody to a surface plasmon resonance (SPR) analysis, wherein the SPR analysis comprises use of extracellular vesicles expressing CD20 as a ligand and the anti-CD20 antibody as an analyte.

21. The method of claim 20, wherein the anti-CD20 antibody is selected from the group consisting of rituximab, ocrelizumab, ofatumumab, obinutuzumab, a CD20 T-Cell dependent bispecific antibody, and combinations thereof.

22. The method of claim 20, further comprising detecting a presence of an extracellular marker, wherein the extracellular marker is selected from the group consisting of CD81, CD63, CD9, and combinations thereof.

23. A method for determining the activation of T cells obtained from a patient comprising
a) incubating extracellular vesicles expressing CD20 with T cells and a CD20 T-cell dependent bispecific antibody, and
b) determining the activation of T cells.

24. The method of claim 23, further comprising detecting a presence of an extracellular marker, wherein the extracellular marker is selected from the group consisting of CD81, CD63, CD9, and combinations thereof.

25. A method of treating a tumor in a subject in need comprising:
a) obtaining a sample from the subject,
b) generating a calibration curve using extracellular vesicles comprising a tumor antigen,
c) comparing a level of the tumor antigen in extracellular vesicles in the sample with the calibration curve to determine the quantity of the target tumor antigen in the extracellular vesicles in the sample,
d) determining whether the subject is likely to exhibit a response to an antibody therapy, based on the level of the tumor antigen in extracellular vesicles in the sample, and
e) administering a therapeutic in response to the determination in d).

26. The method of claim 25, further comprising detecting a presence of an extracellular marker, wherein the extracellular marker is selected from the group consisting of CD81, CD63, CD9, and combinations thereof.

27. The method of claim 25, wherein the antibody is selected from the group consisting of rituximab, ocrelizumab, ofatumumab, obinutuzumab, and combinations thereof.

28. The method of claim 25, wherein the target tumor antigen is selected from the group consisting of a human CD20 antigen, a mouse CD20 antigen, a rat CD20 antigen, a rabbit CD20 antigen, a cynomolgus monkey CD20 antigen, a human CD3 antigen, a mouse CD3, a rat CD3 antigen, a rabbit CD3 antigen, a cynomolgus monkey CD3 antigen, a human FcRH5 antigen, a human Ly6G6 antigen, a human HER2 antigen, a human EGFR antigen, a human HER3 antigen, a human HER4 antigen, a human PSMA antigen, and combinations thereof.

29. The method of claim 25, wherein the sample is selected from the group consisting of a plasma sample, a serum sample, a tissue culture supernatant sample, and combinations thereof.

30. The method of claim 25, wherein a concentration of a circulating tumor antigen and the calibration curve is determined using an immunoassay, an ELISA, and/or a Western Blot.

31. The method of claim 25, further comprising detecting a presence of an extracellular vesicle marker, wherein the extracellular vesicle marker comprises a CD81, CD63, and/or CD9.
